(19) United States
(12) Patent Application Publication
(10) Pub. No.: US 2005/0234073 A1
(43) Pub. Date: Oct. 20, 2005

(54) Methods of inhibiting inflammation

(76) Inventor: Richard S. Blumberg, Chestnut Hill, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/054,931

(22) Filed: Feb. 9, 2005

Related U.S. Application Data
(63) Continuation-in-part of application No. 10/808,052, filed on Mar. 24, 2004.
(60) Provisional application No. 60/457,048, filed on Mar. 24, 2003.

(51) Int. Cl. A61K 31/506; A61K 31/4545; A61K 31/454; A61K 31/427; A61K 31/422; A61K 31/4184

(52) U.S. Cl. 514/256; 514/319; 514/318; 514/326; 514/365; 514/374; 514/384

(57) ABSTRACT

The invention provides a method of inhibiting inflammation in a mammal, by administering to the mammal composition containing a compound which inhibits the expression or activity of a microsomal triglyceride transfer protein.

### RELATED APPLICATIONS

This application is a continuation in part of U.S. Ser. No. 10/808,052 filed Mar. 24, 2004 which claims the benefit of U.S. Ser. No. 60/457,048 filed Mar. 24, 2003 each of which is incorporated herein by reference in its entirety.

### STATEMENT AS TO FEDERALLY SPONSORED RESEARCH

This invention was made with U.S. government support under NIH grants DK44319, DK53056 and DK51362. The government has certain rights in the invention.

### FIELD OF THE INVENTION

The invention relates to methods of modulating the immune response.

### BACKGROUND OF THE INVENTION

The inflammatory response plays an important role in limiting and controlling pathogenic infections. Elevated levels of proinflammatory cytokines are also associated with a number of diseases of autoimmunity such as toxic shock syndrome, rheumatoid arthritis, osteoarthritis, diabetes and inflammatory bowel disease. In these diseases, chronic elevation of inflammation exacerbates or causes much of the pathophysiology observed.

### SUMMARY OF THE INVENTION

The invention is based on the discovery that a decrease in microsomal triglyceride transfer protein (MTP) leads to an inhibition of inflammation in a mouse model for clinical inflammation. In addition, it was discovered that MTP directly transfers lipid to CD1. Accordingly, the invention features methods of preventing or inhibiting inflammation or immune function in a bodily tissue. The inflammation is CD1-mediated inflammation. Inflammation is inhibited by administering to an inflamed tissue (or a tissue that is at risk of becoming inflamed) a MTP inhibitor. An inflamed tissue is characterized by redness, pain and swelling of the tissue. Inhibition of immune function is defined by a decrease in action of the immune system such as a decrease in T-cell activation or antibody production by B-cells in response to an antigen. The tissue includes epithelial tissue or liver tissue. For example, the epithelial tissue is intestinal tissue or skin. The invention also features methods of preventing or alleviating a symptom of an inflammatory disorder or a CD1-mediated immunopathology in a subject by identifying a subject suffering from or at risk of developing an inflammatory disorder or a CD1-mediated immunopathology and administering to the subject a MTP inhibitor. A CD1-mediated immunopathology includes for example an autoimmune disorder such as diabetes, colitis, or hepatitis.

Inflammation or immune function is inhibited by contacting a cell with a MTP inhibitor in an amount that leads to a reduction in the production of a proinflammatory or inflammatory cytokine; in an amount that leads to inhibition of T-cell activation, e.g. activation of a CD1d restricted T cell or in an amount that reduces the transfer of lipid, e.g., a glycolipid, a phospholipid or a lipopeptide from MTP to CD1. The transfer of lipid is direct. By direct lipid transfer it is meant that lipid is transferred to CD1 from MTP without any intermediates or accessory molecules.

The cell is any cell that is capable of expressing MTP, e.g., an antigen presenting cell such as a B-cell, monocyte, macrophage, dendritic cell; a hepatocyte; or an epithelial cell such as an intestinal epithelial cell, a thymocyte or a T-cell. Optimally, the cell expresses CD1 (e.g., CD1a, CD1b, CD1c, CD1d or CD1e), a natural killer receptor, an invariant T-cell receptor (e.g., Vα24Jα15), or any T-cell receptor that is CD1-restricted. For example, the cell overexpresses MTP compared to a level of expression associated the normal non-inflamed tissue or cells. The cell is contacted in vivo, in vitro, or ex vivo. Inflammatory cytokines include for example, interferon (e.g., IFN-γ), interleukin (e.g., IL-2), or tumor necrosis factor alpha.

The invention includes methods of inhibiting antigen presentation or cell surface expression of CD1 (e.g., CD1a, CD1b, CD1c, CD1d or CD1e) by contacting a cell with a MTP inhibitor. CD1 mediated antigen presentation is inhibited such that the amount of lipid associated with CD1 or the amount of binding to CD1 is reduced in the presence of the inhibitor compared to the absence of the inhibitor. The cell is an immune cell such as an antigen presenting cell. The cell expresses CD1. For example, the cell expresses CD1d. The cell is a B-cell, a macrophage, a dendritic cell a hepatocyte an epithelial cell or any cell expressing CD1.

Also included in the invention is a method of inhibiting an association of MTP and CD1 by contacting a cell with an MTP-binding compound or a CD1 binding compound such that the association is reduced in the presence of the compound compared to the absence of the compound. The association is non-covalent such as a van der Walls interaction, hydrogen bonding, electrostatic interaction, or hydrophobic interaction. A MTP binding compound is any compound that interacts (covalently or non-covalently) with an MTP protein. The MTP binding compound decreases the lipid transfer activity of the MTP protein. The compound interacts with the M subunit of an MTP protein. Alternatively, the compound interacts with the P subunit of an MTP protein. For example, the compound interacts with the lipid transfer domain, the membrane associating domain or apoB binding domain of the M subunit. Optimally, the compound interacts with the A or C β-sheets or both of the M subunit. The compound interacts with the amino acid residue at position 780 of the M subunit of a MTP protein. A CD1 binding compound is any compound that interacts with a CD1 polypeptide. The CD1 binding compound prevents lipidation of the CD1 polypeptide.

The compound interacts with the α1, α2, α3 or the β2m domain of a CD1 polypeptide. For example, the compound interacts with residues or regions of the CD1 molecule that share homology with the MTP binding domains on apoB as shown in FIGS. 12-14, e.g., α1 and α2 domains that contain lipid contact sites.

A MTP inhibitor is a compound which decreases the expression or activity of MTP. MTP activities include binding to CD1 and transferring lipid. A MTP inhibitor preferentially associated with a phospholipid binding domain of MTP compared to a triglyceride binding domain of MTP. Optimally, the MTP preferentially reduces transfer of lipid from MTP to CD1 compared to apoB. For example, a lesser amount of lipid, e.g., 10%, 20%, 30%, 40%, 50%, 60%, 70% or less is transferred to CD1 as compared to apoB in the presence of the MTP inhibitor. The MTP inhibitor preferentially reduces transfer of a phospholipid or other CD-1 binding lipid (e.g., lipopeptide, glycolipid, lipoarabino mannans, or monoglucosides) compared to a triglyceride. The inhibitor interacts with the neutral lipid and phospholipid binding site (i.e., the fast site) of MTP. Alternatively, the inhibitor interacts with the phospholipid binding site (i.e., the slow site) of MTP. The MTP inhibitor preferentially interacts with the slow site. Activity of MTP is measured by determining the transfer of lipids from HDL to LDL. For example, a decrease in the transfer of lipid from HDL to LDL in the presence of the compound (compared to the amount detected in the absence of the compound) indicates a reduction of MTP activity. Methods of measuring transfer of lipid are well known in the art.

The subject is a mammal such as human, a primate, mouse, rat, dog, cat, cow, horse, or pig. The subject is suffering from or at risk of developing an inflammatory disorder or a neoplastic disorder. Inflammatory disorders include, cardiovascular inflammation, gastrointestinal inflammation, hepatic inflammation, pulmonary inflammation, autoimmune disorders or skeletal inflammation. A subject suffering from or at risk of developing inflammatory is identified by methods known in the art, e.g., gross examination of tissue or detection of inflammation associated in tissue or blood. Symptoms of inflammation include pain, redness and swelling of the affected tissue. A subject suffering from gastrointestinal inflammation, such as colitis, is identified histologically by the presence of mucosal necrosis or hemorrhagic lesions in the colon, frequent diarrhea or blood and pus in the stool.

MTP inhibitors include compounds that reduce expression of a Mttp gene product. For example, the compound is an antisense MTP nucleic acid, an MTP-specific short-interfering RNA, or a MTP specific ribozyme. Alternatively, the MTP inhibitor is a protease inhibitor, a carboline compound or a benzimidazole-based analogue. MTP inhibitors are administered alone or in combination with another anti-inflammatory agent or therapeutic drugs used to treat an inflammatory disorder. For example, the MTP inhibitor is administered in combination with corticosteroids, cyclosporine, nicotine, or heparin.

Other features and advantages of the invention will be apparent from the following detailed description and from the claims.

### BRIEF DESCRIPTION OF THE DRAWINGS

FIG. 1A is a photograph of a Western blot. Lysates of hepatocytes from wild-type C57BL/6J (B6) mice were immunoprecipiated with control immunoglobulins (rat IgG2b, mouse IgG1), an anti-H-2D antibody (8F12, mouse IgG1), or an anti-CD1d antibody (1B1, rat IgG2b) and the immunoprecipitates Western blotted an anti-MTP antibody (left panel) or a rabbit anti-MHC class I serum (right panel)

FIG. 1B is a photograph of a Western blot. Lysates were prepared from B6 or MTP^{flox/flox}Mx1Cre mice before or after pIpC treatment and subjected to the same analysis as described in panel A.

FIG. 1C is a photograph of hepatocytes isolated from MTP^{flox/flox}Mx1Cre mice prior to treatment with pIpC or MTP^{flox/flox}Mx1Cre mice at one day after completion of pIpC treatment (bottom panels) were stained with the anti-CD1d mAb, 1B1, an anti-ER antibody and a marker for the nuclei. MTP^{flox/flox}Mx1Cre mice that were treated with pIpC were also stained for expression of MHC class I using an H-2K^{b} antibody.

FIG. 1D is an illustration depicting the results of fluorescent activated cell sorting showing a decrease in CD1d surface expression on hepatocytes following deletion of the MTP gene.

FIG. 2A is a bar chart showing induction of mIL-2 secretion by the mouse iNKT cell hybridoma DN32.D3 cocultivated with hepatocytes, obtained from MTP^{flox/flox}Mx1Cre mice treated (+) or not treated (−) with pIpC, in the presence of either αGalCer (+, 100 ng/ml) or vehicle control (−). (n=6 mice per group). ND, not detectable. *, P<0.001.

FIG. 2B is a bar chart showing induction of mIL-2 production by CD11c⁺ liver cells from MTP^{flox/flox}Mx1Cre mice treated with or without pIpC that were cultured for 24 h with CD4⁺ splenocytes from OT-II mice and 100 μg/ml OVA or PBS. In the presence of OVA, the concentration of IL-2 in culture supernatants from CD11c⁺ cells isolated from pIpC-treated mice was significantly increased consistent with an upregulation of MHC class II by interferon-α. **, P<0.005.

FIG. 2C is a bar chart showing induction of mIL-2 production by hepatocytes from MTP^{flox/flox}Mx1Cre mice treated (+) or not treated (−) with pIpC that were cocultivated with the autoreactive mouse CD1d-restricted T cell hybridomas 14S.6 (non-invariant TCR-α chain) or 24.8 (invariant TCR-α chain) (n=2 mice per group, duplicate cultures). CD1d-restriction was confirmed with an anti-CD1d blocking antibody (3C11). Note the reduction in the degree of autoreactivity of the hybridomas towards hepatocytes derived from pIpC treated vs. non-pIpC treated MTP^{flox/flox}Mx1Cre mice and the blockade of autoreactivity with the 3C11mAb. *, P<0.001.

FIG. 2D is a series of bar charts showing serum ALT levels (U/L) (n=3 mice per group) from C57BL/6J mice (panels a and b) or MTP^{flox/flox}Mx1Cre mice (panels c and d) Mice were treated with either vehicle alone (open bars), pIpC alone (closed bars), αGalCer alone (hatched bars) or pIpC followed by αGalCer (shaded bars). Panels a and c show the results when αGalCer was administered at day 1 after completion of the pIpC treatment regimen and panels b and d when αGalCer was administered at day 10 after similar treatment. Similar results were obtained when serum AST levels were assessed (*; P<0.05; **, P<0.01.

FIG. 2E are photographs of gross examination of the livers of C57BL/6J mice (panels a and b) and MTP^{flox/flox}Mx1Cre mice (panels c and d) that received αGalCer with (panels b and d) or without (panels a and c) prior pIpC treatment. Note the multiple foci of liver necrosis in all groups (white arrows) except for the MTP^{flox/flox}Mx1Cre mice that received pIpC treatment at 10 days prior to αGalCer administration (panel d). The latter group of mice only exhibited a pale liver consistent with the lipid accumulation.

FIG. 2F are photographs of histopathologic analysis livers of αGalCer induced hepatitis in C57BL/6J and MTP^{flox/flox}Mx1Cre mice treated as described in FIG. 2C.

FIG. 3A depicts the results of silencing the MTPp gene in intestinal epithelial cells. Panel A are photographs of blots showing MTP transcripts in MODE-K cells relative to hepatocytes. Panel B are photographs of blots showing MTP transcripts in MODE-K cells that were either not treated (−) or treated with mock siRNA oligomers specific for an irrelevant gene target (Src homology domain 2 phosphatase 1, SHP-1; mock) or siRNA oligomers specific for MTP (silenced). β-actin uses as a control. Panel C is a bar chart showing quantitation of the ratios between MTPp and β-actin levels for each of the experimental conditions shown in Panel B. *, P<0.001.

FIG. 3B is a bar chart showing MODE-K cell (treated in triplicate as in FIG. 2A with siRNA oligomers specific for SHP-1 (mock) or MTP) induction of mIL-2 production upon by cocultivation with the DN32.D3 cell line in the presence of αGalCer (+, 100 ng/ml) and mIL-2 production (pg/ml) assessed. **, P<0.01. ND, non-detectable.

FIG. 4A are line graphs showing wasting as defined by % of body weight from baseline of B6 (panel A) or MTP^{flox/flox}Mx1Cre (panel B) of mice subjected to oxazolone colitis treated either with vehicle (squares) or with pIpC (circles) prior to skin painting with oxazolone followed by rectal challenge with either 50% ethanol alone as a control (open squares and circles) or 50% ethanol containing oxazolone (closed circles and squares). Data are shown as mean values+SEM and represent 12 mice per group. *, P<0.005; **, P<0.001 for MTP^{flox/flox}Mx1Cre mice with or without pIpC treatment.

FIG. 4B are photographs of H & E stained microscopic images from oxazolone colitis or ethanol control group in WT B6 or MTP^{flox/flox}Mx1Cre mice treated with vehicle or pIpC are shown (magnification; ×100). One representative picture from each group is shown. (Panel A, B6 mice with oxazolone+vehicle; Panel B, B6 mice with oxazolone+pIpC; Panel C, MTP^{flox/flox}Mx1Cre mice with oxazolone+vehicle; and Panel D, MTP^{flox/flox}Mx1Cre mice with oxazolone+pIpC). Black arrow in Panel D indicates mild inflammation with minimal epithelial cell proliferation in MTP^{flox/flox}Mx1Cre mice treated with pIpC in comparison to the untreated control group (Panel C).

FIG. 4C are bar charts showing the quantitative histopathological assessment of oxazolone colitis activity in B6 (Panel A) or MTP^{flox/flox}Mx1Cre (Panel B) mice from the following groups are shown: ethanol control, shaded bars; oxazolone, open bars; ethanol with pIpC, hatched bars; oxazolone with pIpC, filled bars. Note the significant suppression of oxazolone colitis in MTP^{flox/flox}Mx1Cre but not B6 mice pretreated with pIpC (filled bars) in comparison to vehicle treated animals (open bars)*; P<0.001. Data are shown as mean values±SEM and represent ten mice per group.

FIG. 5 is a diagram illustrating an experimental model for reduction of oxazolone-induced colitis in MTP deficient mice.

FIG. 6 is a photograph of a Western Blot showing MTP expression in antigen presenting cell.

FIG. 7 is a bar chart showing antigen presentation of siRNA silenced and wild type U397 cells at an effecter target ration of 1:1.

FIG. 8 is a bar chart showing antigen presentation of siRNA silenced and wild type U397 cells at an effecter target ration of 10:1.

FIG. 9 is a bar chart showing antigen presentation of siRNA silenced and wild type U397 cells at an effecter target ration of 100:1.

FIG. 10 is an illustration depicting types of natural killer T-cells.

FIG. 11 is an illustration depicting the regulatory functions of iNKT cells.

FIG. 12 is an illustration depicting a sequence alignment of the low affinity MTP binding region (amino acids 1-269) of apoB.

FIG. 13 is an illustration depicting a sequence alignment of the high affinity MTP binding region (amino acids 512-721) of apoB.

FIG. 14 is an illustration depicting a sequence alignment of the high affinity MTP binding region (amino acids 270-570) of apoB.

FIG. 15A is a photograph of a RT-PCR reaction products showing MTP is present in antigen presenting cells. MTPA dendritic cells were obtained from the liver of a pIpC-injected MTPmx1 mouse.

FIG. 15B is a photograph of a Western Blot showing MTP is present in splenocyte microsomes (flt3L splen micro) and mitochondria (flt3L splen mito).

FIG. 15C is a line graph showing triglyceride transfer activity in 100 ug splenocyte lysate (Δ), 50 μg splenocyte lysate (◯), 100 μg splenocyte lysate with BMS197636 (▴), and 50 μg splenocyte lysate with BMS197636 (●). Assays were in triplicate with standard deviations less than 10% of percent transfer at 3 hours.

FIG. 16A is a bar chart showing purified MTP transfers a lipid to recombinant CD1d in vitro. Two micrograms of BSA or recombinant CD1d were coated onto microtiter wells and incubated with either PE:NBD vesicles alone or PE:NBD vesicles plus 0.5 μg purified MTP. Results are representative of five independent experiments. *, p<0.005

FIG. 16B is a graph showing purified MTP transfers a lipid to recombinant CD1d in vitro. (b) Two micrograms of recombinant CD1d were coated onto microtiter wells and incubated with PE:NBD vesicles and increasing concentrations of purified MTP. *, p<0.005; **, p<0.001 compared to 0 μg MTP.

FIG. 17A is a bar chart showing IL-2 production of MODE-K cells cultured in the presence of BMS212122 (MTPi) or vehicle were incubated with α-galcer for 3 hours, washed, and co cultured with DN32 cells. IL-2 concentrations were measured by ELISA of 24 hour culture supernatants.

FIG. 17B is a series of bar charts showing IL-2 production of splenocytes isolated from wild type mice in the presence or absence of exogenous antigens. Splenocytes were incubated for 24 hours with BMS212122 and washed. Exogenous antigens ovalbumin or α-galcer was added as indicated. Splenocytes were then co cultured with: CD4⁺ cells from an OT-II transgenic mouse (left); DN32 NKT cells (center); or autoreactive 24.8 NKT cells (right). IL-2 concentrations were measured by ELISA of 40 hour culture supernatants. *, p<0.05.

FIG. 17C is a bar chart showing IL-2 production of splenocytes or bone marrow derived dendritic cells co cultured with 24.8 NKT cells and incubated for 24 hours with BMS212122. Anti-CD1d mAb 1B1 or an isotype control was added as indicated. IL-2 concentrations were measured by ELISA of 40 hour culture supernatants. (nd=not detected) *, p<0.02.

FIG. 17D is a line graph showing IL-2 production in bone marrow derived dendritic cells incubated for 4 days with BMS212122 (◯) or vehicle (●) were treated with α-galcer for 3 hours, washed and co cultured with DN32. IL-2 concentrations were measured by ELISA of 24 hour culture supernatants. *, p<0.01.

FIG. 17E is a flow cytometric analysis showing BMDCs cultured for 4 days with BMS212122 were stained with FITC-conjugated mAb 1B1. Dashed line represents the isotype control.

FIG. 17F is a bar chart showing 11-2 production of CD11c⁺ splenocytes incubated for 24 hours with BMS212122 or bone marrow derived dendritic cells incubated for 3 days with BMS212122 were washed and co cultured with 100 μg/mL ovalbumin and 100,000 CD4⁺ cells from an OT-II transgenic mouse. IL-2 concentrations were measured by ELISA of 24 hour culture supernatants.

FIG. 18 is flow cytometric analysis of in vitro differentiated bone marrow cells from wild type mice incubated with BMS212122 (MTPi) or vehicle Surface staining was performed using antibodies to MHC II (FITC-conjugated) and CD11c (PE-conjugated).

FIGS. 19A and B are bar charts showing dendritic cells isolated from liver or spleen from MTP gene deleted mice have reduced CD1d antigen presentation. (top panels) CD11c⁺ cells were incubated with α-galcer or vehicle for 3 hours, washed and co cultured with DN32 cells (effector:target (E:T)=1:2). IL-2 concentrations were measured by ELISA of 24 hour culture supernatants. *, p<0.05 (middle panels). CD11c⁺ cells at indicated concentration of APCs were co cultured with autoreactive 24.8 NKT cells. IL-2 concentrations were measured by ELISA of 24 hour culture supernatants. *, p<0.05 (bottom panels). CD11c⁺ cells were co cultured with ovalbumin or vehicle and CD4⁺ cells from an OT-II transgenic mouse (E:T=5:1). IL-2 concentrations were measured by ELISA of 24 hour culture supernatants. *, p<0.01.

FIG. 20A is a photograph of RT-PCR reaction products showing MTP expression in U937 cells treated with irrelevant or MTP-specific siRNA oligomers. RNA was isolated 48 hours post silencing and transcript levels of mttp and β-actin were determined by RT-PCR.

FIG. 20B is a bar chart showing IL-2 production in silenced and mock silenced U937 cells incubated with α-galcer for 3 hours. IL-2 concentrations were measured by ELISA of 24 hour culture supernatants. *, p<0.001 (ND=none detected).

FIG. 20C is a bar chart showing IL-2 production in U937 cells cultured with BMS212122 or vehicle for 3 days that were incubated with α-galactosylceramide (α-galcer). IL-2 concentrations were measured by ELISA of 24 hour culture supernatants. *, p<0.001.

FIG. 20D is a bar chart showing IL-2 production in C1Rd cells cultured with BMS212122 or vehicle for 5 days that were incubated with α-galcer for 3 hours IL-2 concentrations were measured by ELISA of 24 hour culture supernatants. *, p<0.02.

FIG. 20E is a line chart showing IFN-gamma production in C1Rd cells cultured with BMS197636 or vehicle and incubated with NKT cell lines derived from human peripheral blood in the presence of 1 ng/mL PMA.

FIG. 20F is a series of flow cytometric analysis showing surface staining of human monocyte-derived DCs were stained with 42.1 (CD1d), anti-HLA-A,B,C (MHC class I), or isotype control antibodies (dashed lines) after 4 days of differentiation in the presence of BMS212122 (gray lines) or vehicle control (black lines).

FIG. 20G is a line graph showing IL-2 production in human moDCs differentiated in the presence of BMS212122 (◯) or vehicle (●) were incubated with α-galcer. IL-2 concentrations were measured by ELISA of 24 hour culture supernatants. *, p<0.05; **, p<0.01.

FIG. 21 is a series of flow cytometric analysis showing surface staining of CD14⁺ monocytes that were differentiated to dendritic cells in the presence of BMS212122.

FIG. 22 is a series of line graphs showing IFN gamma production in CD14⁺ monocytes that were differentiated to dendritic cells in the presence of BMS212122.

FIG. 23 is a series of line graphs showing IFN gamma production in CD1a, CD1c or mock transfected C1R cells cultures with BMS212122.

FIG. 24 is a schematic representation of various MTP inhibitors illustrating their structural similarities.

### DETAILED DESCRIPTION

The invention is based in part on the discovery that a decrease in microsomal triglyceride transfer protein (MTP) expression results in an anti-inflammatory effect. More specifically, it was discovered that MTP directly transfers lipid to CD1.

MTP, an endoplasmic reticulum (ER) resident protein in hepatocytes and intestinal epithelial cells (IEC) is essential for lipidation of apolipoprotein-B (apo-B). Specifically, MTP catalyzes the transport of triglyceride (TG), cholesteryl ester (CE), and phosphatidylcholine (PC) between small unilamellar vesicles (SUV). MTP is a complex of two subunits of molecular weights 58,000 (the “P” subunit) and 88,000 (the “M” subunit. The P subunit is inactive with respect to its isomerase activity in the MTP complex. The P subunit is not essential for its lipid transfer activity, whereas the M subunit is essential for the transfer of lipid. MTP contains three structural motifs (i.e., N-terminal β-barrel, central α-helix, and C-terminal lipid cavity) and three functional domains (i.e., lipid transfer, membrane associating and apoB binding). The lipid transfer domain is involved in loading and unloading of lipid molecules necessary for lipid transfer. The lipid binding domain are formed by the A and C β-sheets of the M subunit. A non-sense mutation in the A sheet at amino acid residue 780 (Asn-Tyr) abolishes MTP's lipid binding activity. In addition, to the lipid transfer activity function, MTP has been shown to physically interact with apoB and this association is important in the regulation of lipoprotein production. However, the lipid transfer domain is different from the apo-B binding domain as lipid transfer inhibitors do not inhibit apo-B-MTP binding.

CD1 is a family of nonpolymorphic cell surface glycoproteins encoded outside the major histocompatibility complex (MHC) but with distant relationship to both MHC class I and class II molecules. The CD1 family is divided into two groups by sequence homology. Group I, consists of CD1a, -b, and -c isotopes and Group II contains CD1d and CD1e. The human group I CD11a, b, and c proteins present mycobacterial lipids and lipopeptides but can also present host lipids to autoreactive CD1 restricted T cells. The intracellular localization of CD1 proteins is controlled by dileucine and tyrosine sorting motifs in their cytoplasmic tails. The type of lipid each CD1 family member presents reflects both the shape of the CD1 hydrophobic antigen binding pocket and the endosomal compartments through which the CD1 proteins traffic. Group II CD1d, found in humans and the only CD1 protein in rodents, presents glycolipid antigens to natural killer T (NKT) cells. Several host lipids have been proposed to associate with CD1d and activate subsets of NKT cells including phosphatidyl inositol (PI), phosphatidyl ethanolamine (PE), isoglobotrihexosylceramide (iGb3), which is required for NKT cell development. Activation of autoreactive NKT cells by CD1d presenting host lipids is beneficial during bacterial and viral infections, some anti-tumor responses and during regulation of autoimmune diseases such as diabetes However, improper activation of NKT cells lead to immune mediated inflammatory disorders such as inflammatory bowel disease, dermatitis, asthma, systemic lupus erythematosus and atherosclerosis, anti-tumor immunity, and anti-microbial immunity.

The ER chaperones calnexin, calreticulin and ERp57 associate with nascent CD1d and assist in folding and disulfide bond formation. Unlike MHC class I, which associates with β2-microglobulin (β2m) early in biogenesis, CD1d acquires β2m just prior to exiting the ER, and β2m is not essential for CD1d cell surface expression. Phospholipids bind to the hydrophobic pocket of nascent CD1d and enable proper folding in a manner analogous to that of peptide in MHC class I assembly. The ER phospholipids bound to CD1d may be replaced when CD1d recycles into endosomal compartments, and recent work on lysosomal saposins has shown that this family of lipid transfer proteins is capable of exchanging or editing the lipid cargo of CD1d. In mice, saposin B can load iGb3 onto CD1d which then activates invariant Vα14 NKT cells. Tail deleted forms of CD1d that fail to traffic to endosomes activate Vα14⁻ NK1.1⁻ NKT cells but cannot present antigen to invariant NKT cells. Furthermore, mice which express only the tail-deleted form of CD1d support thymic development of diverse but not invariant NKT cells indicating a distinction in the host lipids recognized by these two NKT cell populations.

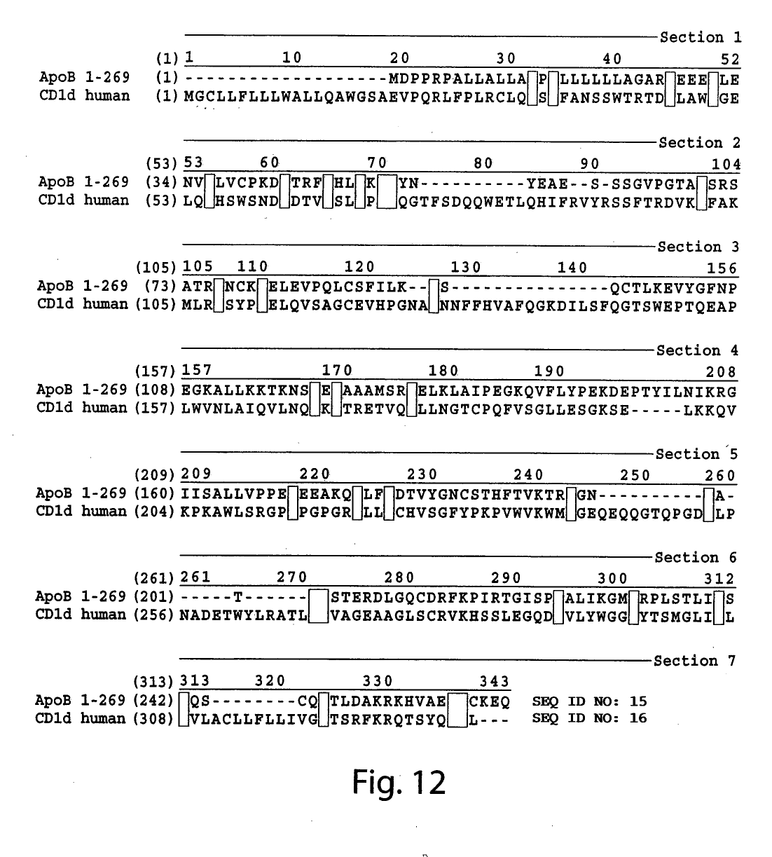

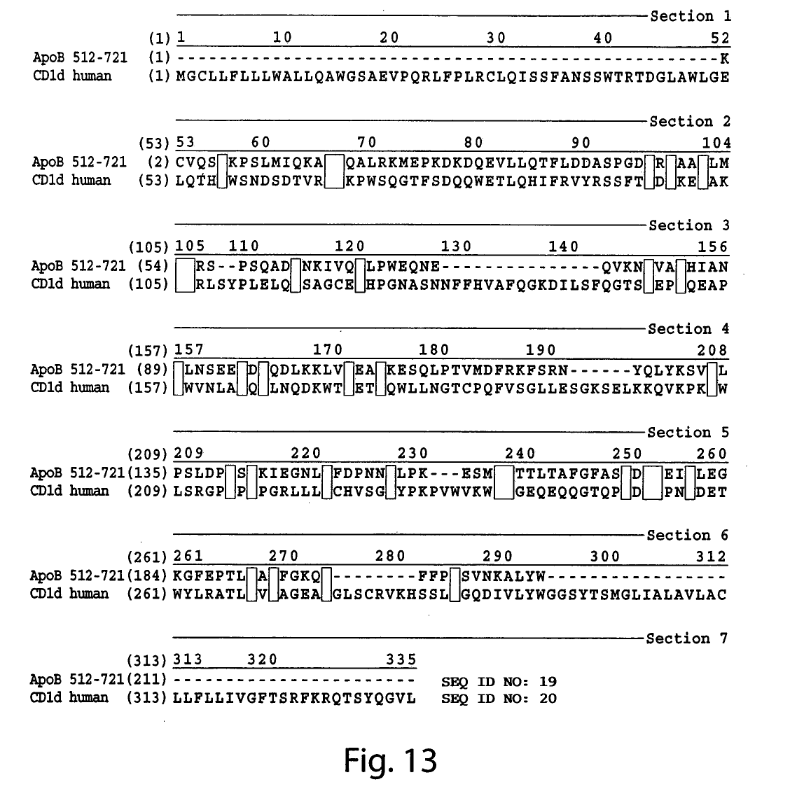

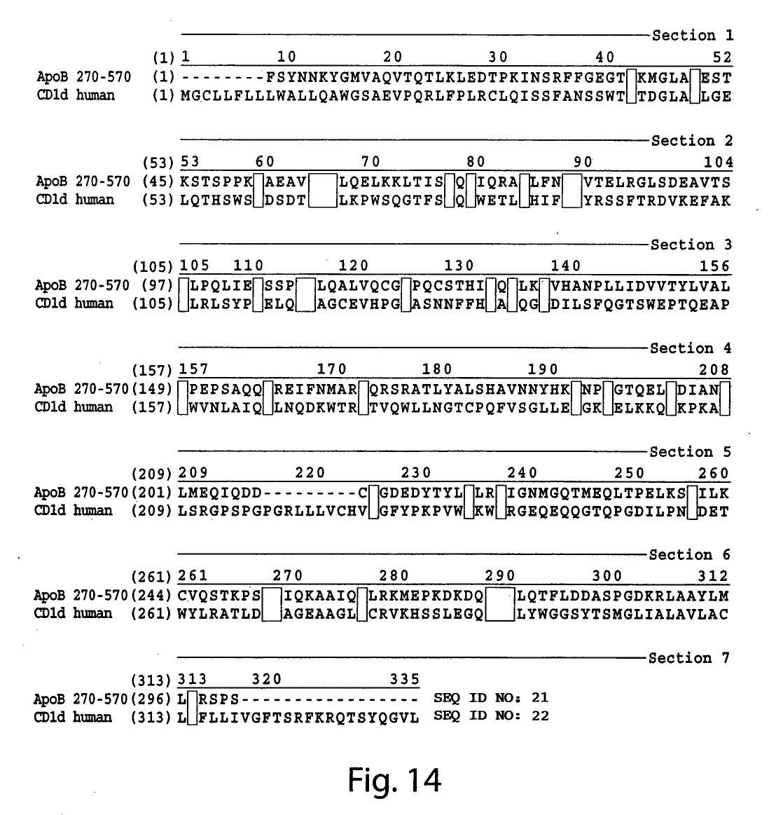

The CD1 molecule comprises four domains; α1, α2, α3 and β2m. MTP interacts with three region on apoB, one low affinity binding region corresponding to amino acid residues 1-269, and two high affinity binding regions corresponding to amino acids 270-570 and 512-721. Sequence aligment of these three MTP binding domains of apoB with CD1d identified regions throughout the CD1d molecule that align with these regions of apoB. (FIGS. 12-14) The analysis indicates that α1, α2 and α3 domains participate the lipid binding activity of CD1. Contacting these CD1 with a soluble compound that binds to or associates with one or more of these domains inhibits an CD1 activity, e.g. binding to MTP.

Hepatocytes from animals in which the Mttp gene has been conditionally deleted and IECs in which Mtpp gene products have been silenced by siRNA fail to activate iNKT cells. Conditional deletion of the Mttpp gene is associated with a redistribution of CD1d expression in hepatocytes and resistance of Mttp gene deleted mice to immunopathologies associated with iNKT mediated hepatitis and colitis. In vivo studies using a conditional MTP knockout mouse model demonstrated that deletion of the Mttp gene conferred protection of oxazolone-induced colitis. This result was surprising, as it would be expected that deletion of the Mttp gene would increase the sensitivity to tissue exposed to toxins. Chemical inhibition of the Mttp gene also results in diminished CD1d-restricted presentation of exogenous and endogenous antigens to NKT cells. In addition, MTP chemical inhibition or deletion in mouse and human primary antigen presenting cells results in a significant reduction in the surface expression of CD1d. Specifically, small molecules that specifically inhibit MTP-mediated lipid transfer and lipidation of apoB are also capable of inhibiting MTP-mediated regulation of CD1d function. Blockade of MTP lipid transfer function with 9-[4-[[[4′-(trifluoromethyl)[1,1′-biphenyl]-2-yl]carbonyl]amino]butyl-piperidin-4-yl]-N-(2,2,2-trifluoroethyl)-9H-fluorene-9-carboxamide (i.e., BMS 197636) or 9-[4-[2,5-Dimethyl-4-[[[4′-(trifluoromethyl)[1,1′-biphenyl]-2-yl]carbonyl]amino]-1H-benzimidazol-1-yl]butyl]-N-(2,2,2-trifluoroethyl)-9H-fluorene-9-carboxamide (i.e., BMS 212122) suppresses CD1d-restricted presentation of exogenous and endogenous antigens by mouse and human professional antigen presenting cells. Notably, presentation via MHC class II is unaffected by the absence of MTP indicating that lack of MTP specifically affects CD1d function. These studies indicate that MTP regulates the ability of CD1d-bearing cell types to exhibit CD1d-restricted antigen presentation.

It has also been demonstrated herein that MTP transfer phospholipid antigens directly to CD1d in vitro. Prior to this discovery, apoB was the only known recipient of MTP transferred lipids. Importantly, apoB transcripts in MTP-positive antigen presenting cells were not detected. This is thus the first evidence of MTP expression in the absence of lipoprotein production, suggesting that the ER-resident MTP serves as a chaperone during CD1d biogenesis and is responsible for transferring lipids to the nascent CD1d pocket. The types of lipids loaded initially onto CD1d could affect the ability of the CD1d antigen to be edited by saposins or other endosomal lipid transfer proteins. MTP is known to transfer a wide range of lipids, kinetics studies have shown that triglycerides are the preferred substrate and are transferred much faster than phospholipids. These suggest at least two different lipid binding sites on MTP, one site that binds and transfers lipids to apoB and another site that binds and transfers lipids to CD1. The ability of MTP to transfer lipids depends on the number and length of the lipid acyl chains and does not depend on the head group as MTP has been shown to transfer all types of phospholipids tested with equal efficacy. Recent work has shown that lysosomal saposins edit the antigens presented on CD1d by replacing presumably ER-derived lipids with lipids present in endosomes and lysosomes.

These results indicate that, similar to the relationship between MTP and apo-B, MTP is involved in the direct lipidation and functional maturation of CD1 This functional maturation of CD1 controlled by MTP includes the acquisition of glycolipid antigens involved in the normal function of CD1 in vivo and indicates that blockade of MTP function will be of therapeutic benefit in diseases mediated by CD1 and related pathways. Specifically, inhibition of CD1 function by the use of small molecule inhibitors, provides therapeutic benefits in inflammatory disorders, such as in ulcerative colitis, lupus, atherosclerosis, and airway hypersensitivity, and in disorders associated with CD1d-mediated antigen presentation.

MTP Inhibitors

A MTP inhibitor is a compound that decreases expression or activity of MTP. A decrease in MTP expression or activity is defined by a reduction of a biological function of the MTP protein. A MTP biological function includes for example, the catalysis of lipid molecules between phospholipid membranes or the transfer of lipid from high density lipoproteins (HDL) to low density lipoproteins (LDL). MTP expression is measured by detecting a MTP transcript or protein. MTP inhibitors are known in the art or are identified using methods described herein. For example, a MTP inhibitor is identified by detecting a decrease the MTP-mediated transfer of lipids from HDL to LDL. Transfer of lipid is detected by methods known in the art such as nuclear magnetic resonance (NMR), electron spin resonance (ESR), radiolabeling or fluorescent labeling. For example, a decrease in transfer of lipid from HDL to LDL in the presence of the compound compared to the absence of the compound indicates a decrease in MTP activity. An MTP inhibitor is also identified by detecting the inhibition of the interaction between CD1 and MTP.

The MTP inhibitor is for example an antisense MTP nucleic acid, a MTP-specific short-interfering RNA, or a MTP-specific ribozyme. Exemplary nucleic acids and polypeptides encoding MTP include for example a human MTP available as GENBANK™ Accession No. NM000253 (SEQ ID NO:3 and SEQ ID NO: 4); Tables 1 and 2) or a murine MTP available as GENBANK™ Accession No. NM008642 (SEQ ID NO:5 and SEQ ID NO: 6; Tables 3 and 4). Start and stop codons are identified in bold in the nucleic acid sequences shown below.

TABLE 1
Human MTP Nucleic Acid (M subunit) (SEQ ID NO:3)

| | |
|---|---|
| actccctcac tggctgccat tgaaagagtc cacttctcag tgactcctag ctgggcactg | 61 |
| gatgcagttg aggattgctg gtcaatatga ttcttcttgc tgtgcttttt ctctgcttca | 121 |
| tttcctcata ttcagcttct gttaaaggtc acacaactgg tctctcatta aataatgacc | 181 |
| ggctgtacaa gctcacgtac tccactgaag ttcttcttga tcggggcaaa ggaaaactgc | 241 |
| aagacagcgt gggctaccgc atttcctcca acgtggatgt ggccttacta tggaggaatc | 301 |
| ctgatggtga tgatgaccag ttgatccaaa taacgatgaa ggatgtaaat gttgaaaatg | 361 |
| tgaatcagca gagaggagag aagagcatct tcaaaggaaa aagcccatct aaaataatgg | 421 |
| gaaaggaaaa cttggaagct ctgcaaagac ctacgctcct tcatctaatc catggaaagg | 481 |
| tcaaagagtt ctactcatat caaaatgagg cagtggccat agaaaatatc aagagaggtc | 541 |
| tggctagcct atttcagaca cagttaagct ctggaaccac caatgaggta gatatctctg | 601 |
| gaaattgtaa agtgacctac caggctcatc aagacaaagt gatcaaaatt aaggccttgg | 661 |
| attcatgcaa aatagcgagg tctggattta cgaccccaaa tcaggtcttg ggtgtcagtt | 721 |
| caaaagctac atctgtcacc acctataaga tagaagacag ctttgttata gctgtgcttg | 781 |
| ctgaagaaac acacaatttt ggactgaatt tcctacaaac cattaagggg aaaatagtat | 841 |
| cgaagcagaa attagagctg aagacaaccg aagcaggccc aagattgatg tctggaaagc | 901 |
| aggctgcagc cataatcaaa gcagttgatt caaagtacac ggccattccc attgtggggc | 961 |
| aggtcttcca gagccactgt aaaggatgtc cttctctctc ggagctctgg cggtccacca | 1021 |
| ggaaatacct gcagcctgac aacctttcca aggctgaggc tgtcagaaac ttcctggcct | 1081 |
| tcattcagca cctcaggact gcgaagaaag aagagatcct tcaaatacta aagatggaaa | 1141 |
| ataaggaagt attacctcag ctggtggatg ctgtcacctc tgctcagacc tcagactcat | 1201 |
| tagaagccat tttggacttt ttggatttca aaagtgacag cagcattatc ctccaggaga | 1261 |
| ggtttctcta tgcctgtgga tttgcttctc atcccaatga agaactcctg agagccctca | 1321 |
| ttagtaagtt caaaggttct attggtagca gtgacatcag agaaactgtt atgatcatca | 1381 |
| ctgggacact tgtcagaaag ttgtgtcaga atgaaggctg caaactcaaa gcagtagtgg | 1441 |
| aagctaagaa gttaatcctg ggaggacttg aaaaagcaga gaaaaaagag gacaccagga | 1501 |
| tgtatctgct ggctttgaag aatgccctgc ttccagaagg catcccaagt cttctgaagt | 1561 |
| atgcagaagc aggagaaggg cccatcagcc acctggctac cactgctctc cagagatatg | 1621 |
| atctcccttt cataactgat gaggtgaaga agaccttaaa cagaatatac caccaaaacc | 1681 |
| gtaaagttca tgaaaagact gtgcgcactg ctgcagctgc tatcatttta aataacaatc | 1741 |
| catcctacat ggacgtcaag aacatcctgc tgtctattgg ggagcttccc caagaaatga | 1801 |
| ataaatacat gctcgccatt gttcaagaca tcctacgttt ggaaatgcct gcaagcaaaa | 1861 |
| ttgtccgtcg agttctgaag gaaatggtcg ctcacaatta tgaccgtttc tccaggagtg | 1921 |
| gatcttcttc tgcctacact ggctacatag aacgtagtcc ccgttcggca tctacttaca | 1981 |
| gcctagacat tctctactcg ggttctggca ttctaaggag aagtaacctg aacatctttc | 2041 |
| agtacattgg gaaggctggt cttcacggta gccaggtggt tattgaagcc caaggactgg | 2101 |
| aagccttaat cgcagccacc cctgacgagg gggaggagaa ccttgactcc tatgctggta | 2161 |
| tgtcagccat cctctttgat gttcagctca gacctgtcac ctttttcaac ggatacagtg | 2221 |
| atttgatgtc caaaatgctg tcagcatctg gcgaccctat cagtgtggtg aaaggactta | 2281 |
| ttctgctaat agatcattct caggaacttc agttacaatc tggactaaaa gccaatatag | 2341 |
| aggtccaggg tggtctagct attgatattt caggtgcaat ggagtttagc ttgtggtatc | 2401 |
| gtgagtctaa aacccgagtg aaaaataggg tgactgtggt aataaccact gacatcacag | 2461 |
| tggactcctc ttttgtgaaa gctggcctgg aaaccagtac agaaacagaa gcaggcttgg | 2521 |
| agtttatctc cacagtgcag ttttctcagt acccattctt agtttgcatg cagatggaca | 2581 |
| aggatgaagc tccattcagg caatttgaga aaaagtacga aaggctgtcc acaggcagag | 2641 |
| gttatgtctc tcagaaaaga aaagaaagcg tattagcagg atgtgaattc ccgctccatc | 2701 |
| aagagaactc agagatgtgc aaagtggtgt ttgcccctca gccggatagt acttccagcg | 2761 |
| gatggttttg aaactgacct gtgatatttt acttgaattt gtctccccga aagggacaca | 2821 |
| atgtggcatg actaagtact tgctctctga gagcacagcg tttacatatt tacctgtatt | 2881 |
| taagattttt gtaaaaagct acaaaaaact gcagtttgat caaatttggg tatatgcagt | 2941 |
| atgctaccca cagcgtcatt ttgaatcatc atgtgacgct ttcaacaacg ttcttagttt | 3001 |
| acttatacct ctctcaaatc tcatttggta cagtcagaat agttattctc taagaggaaa | 3061 |
| ctagtgtttg ttaaaaacaa aaataaaaac aaaaccacac aaggagaacc caattttgtt | 3121 |
| tcaacaattt ttgatcaatg tatatgaagc tcttgatagg acttccttaa gcatgacggg | 3181 |
| aaaaccaaac acgttcccta atcaggaaaa aaaaaaaaaa aaaaaagtaa gacacaaaca | 3241 |
| aaccattttt ttctcttttt ttggagttgg gggcccaggg agaagggaca aggcttttaa | 3301 |
| aagacttgtt agccaacttc aagaattaat atttatgtct ctgttattgt tagttttaag | 3361 |
| ccttaaggta gaaggcacat agaaataaca tc | |

TABLE 2
Human MTP Amino Acid Sequence (SEQ ID NO:4)

MILLAVLFLCFISSYSASVKGHTTGLSLNNDRLYKLTYSTEVLL
DRGKGKLQDSVGYRISSNVDVALLWRNPDGDDDQLIQITMKDVNVENVNQQRGEKSIF
KGKSPSKIMGKENLEALQRPTLLHLIHGKVKEFYSYQNEAVAIENIKRGLASLFQTQL
SSGTTNEVDISGNCKVTYQAHQDKVIKIKALDSCKIARSGFTTPNQVLGVSSKATSVT
TYKIEDSFVIAVLAEETHNFGLNFLQTIKGKIVSKQKLELKTTEAGPRLMSGKQAAAI
IKAVDSKYTAIPIVGQVFQSHCKGCPSLSELWRSTRKYLQPDNLSKAEAVRNFLAFIQ
HLRTAKKEEILQILKMENKEVLPQLVDAVTSAQTSDSLEAILDFLDFKSDSSIILQER
FLYACGFASHPNEELLRALISKFKGSIGSSDIRETVMIITGTLVRKLCQNEGCKLKAV
VEAKKLILGGLEKAEKKEDTRMYLLALKNALLPEGIPSLLKYAEAGEGPISHLATTAL
QRYDLPFITDEVKKTLNRIYHQNRKVHEKTVRTAAAAIILNNNPSYMDVKNILLSIGE
LPQEMNKYMLAIVQDILRLEMPASKIVRRVLKEMVAHNYDRFSRSGSSSAYTGYIERS
PRSASTYSLDILYSGSGILRRSNLNIFQYIGKAGLHGSQVVIEAQGLEALIAATPDEG
EENLDSYAGMSAILFDVQLRPVTFFNGYSDLMSKMLSASGDPISVVKGLILLIDHSQE
LQLQSGLKANIEVQGGLAIDISGAMEFSLWYRESKTRVKNRVTVVITTDITVDSSFVK
AGLETSTETEAGLEFISTVQFSQYPFLVCMQMDKDEAPFRQFEKKYERLSTGRGYVSQ
KRKESVLAGCEFPLHQENSEMCKVVFAPQPDSTSSGWF

TABLE 3
Mouse MTP Nucleic Acid (SEQ ID NO:5)

| | |
|---|---|
| ctggatgtgg cagagggagc cagcatgatc ctcttggcag tgctttttct ctgcttcttc | 61 |
| tcctcctact ctgcttccgt taaaggtcac acaactggcc tctcattaaa taatgagcgg | 121 |
| ctatacaagc tcacgtactc cactgaagtg tttcttgatg ggggcaaagg aaaaccgcaa | 181 |
| gacagcgtgg gctacaaaat ctcatctgat gtggacgttg tgttactgtg gaggaatcct | 241 |
| gatggtgatg atgatcaagt gatccaagtc acgataacag ctgttaacgt tgaaaatgcg | 301 |
| ggtcaacaga gaggcgagaa gagcatcttc cagggcaaaa gtacacctaa gatcataggg | 361 |
| aaggacaacc tggaggctct gcagagaccc atgcttcttc atctggtccg ggggaaggtc | 421 |
| aaggagttct actcctatga aaacgagcca gtgggcatag aaaatctcaa gagaggcttg | 481 |
| gctagcttat tccagatgca gctaagctct ggaactacca acgaggtaga tatctctggg | 541 |
| gattgtaaag tgacctacca ggcccaacaa gacaaagtgg tcaaaattaa ggctctggat | 601 |
| acatgcaaaa ttgagcggtc tggatttaca acggcaaacc aggtgctggg cgtcagttca | 661 |
| aaagccacat ctgtcactac ctacaagata gaggacagct ttgtcaccgc tgtgcttgca | 721 |
| gaagagacca gggcttttgc cttgaacttc caacaaacca tagcaggaaa aatagtgtca | 781 |
| aagcagaaat tggagctgaa gacaactgaa gccggcccaa ggatgatccc cgggaagcaa | 841 |
| gtggcaggtg taattaaagc agttgattcc aaatacaaag ccattcccat tgtgggacag | 901 |
| gtcctcgagc gtgtctgcaa aggatgccct tctctggcgg agcactggaa gtccatcaga | 961 |
| aagaacctgg agcctgaaaa cctgtccaag gccgaggctg tccagagctt cctggccttc | 1021 |
| atccagcacc tccggacttc gaggagagaa gagatcctcc agattctgaa ggcagagaag | 1081 |
| aaagaagtgc tccctcagct ggtggatgcc gtcacctctg ctcagactcc agactcgcta | 1141 |
| gaagccatcc tggacttttt ggatttcaaa agtgacagca gtatcatact ccaggaaagg | 1201 |
| ttcctctatg cctgtggctt tgccacccac cctgatgaag aactcctacg agccctcctt | 1261 |
| agtaagttca aaggttcctt tgcaagcaac gacatcagag agtcggttat gatcatcatt | 1321 |
| ggagccctag tcaggaagct gtgtcagaat gaaggctgca agctcaaggc agtggtggaa | 1381 |
| gctaagaagc tgatcctggg aggacttgaa aaaccagaga agaaagaaga caccacaatg | 1441 |
| tacctgctgg ccctgaagaa tgccttgctt cccgaaggca tcccgctcct tctgaagtat | 1501 |
| gctgaggctg gagaagggcc cgtcagccac ctggccacca ctgttctcca gagatacgat | 1561 |
| gtctccttca tcacagatga ggtgaagaag accttgaaca ggatatacca ccagaatcgt | 1621 |
| aaggttcatg agaagacggt gcgcacaact gccgctgctg tcatcttaaa gaacccatcc | 1681 |
| tacatggatg tgaagaacat cctgctgtcc attggggaac tcccgaaaga gatgaacaaa | 1741 |
| tacatgctca ccgttgtgca agacatcctg cattttgaaa tgcctgcaag caaaatgatc | 1801 |
| cgtcgagttc tcaaggagat ggctgttcac aattatgacc gtttctccaa gagtggatcc | 1861 |
| tcttctgcct atactggcta cgtagaacgt agcccccgtg cagcgtccac atacagcctt | 1921 |
| gacatccttt actctggctc tggcattctg aggagaagta acctgaacat cttccagtac | 1981 |
| atcaaaggaa cagagcttca tggtagtcag gtggtgattg aagcccaagg gctggaaggc | 2041 |
| ttaattgcag ccactcctga tgaaggagag gagaaccttg actcttatgc tggcatgtca | 2101 |
| gccatcctgt ttgatgttca gcttaggcct gtcacatttt ttaatggata cagtgatttg | 2161 |
| atgtccaaaa tgctgtcggc atccggcgac cctgtcagcg tggtgaaagg gcttattctg | 2221 |
| ttaatagacc attctcagga tattcagctg caatctggac taaaggccaa tatggagatc | 2281 |
| cagggtggtc tagctattga tatttctggt tcaatggaat tcagtctgtg gtatcgcgag | 2341 |
| tctaaaaccc gagtgaaaaa tcgggtggct gtggtgataa ccagcgacgt cacagtggat | 2401 |
| gcctcttttg tgaaagctgg tctggaaagc agagcggaga cagaggctgg gctggagttc | 2461 |
| atctccacag tgcagttctc acagtacccg ttcttggtct gcatgcagat ggacaaggct | 2521 |
| gaagccccac tcaggcaatt cgagacaaag tatgaaaggc tatctacagg caggggatat | 2581 |
| gtctctcgga gaagaaaaga gagcctagtg gccggatgtg aactccccct ccatcaacag | 2641 |
| aactctgaga tgtgcaacgt ggtattccca cctcagccag aaagcgataa ctccggtgga | 2701 |
| tggttttgat tcccgtgggt tcccttccac cagaacgata tgctatgacg tgcctgaccc | 2761 |
| ttgctctctg agagcacagt gtttacatat ttacctgtat ttaagatgtt tgtaaagagc | 2821 |
| agtggagaac ttcagttgat taaagttgaa cctattcagg agaagaccca cagtgtcc | |

TABLE 4
Mouse MTP Amino Acid Sequence (SEQ ID NO:6)

MILLAVLFLCFFSSYSASVKGHTTGLSLNNERLYKLTYSTEVFL
DGGKGKPQDSVGYKISSDVDVVLLWRNPDGDDDQVIQVTITAVNVENAGQQRGEKSIF
QGKSTPKIIGKDNLEALQRPMLLHLVRGKVKEFYSYENEPVGIENLKRGLASLFQMQL
SSGTTNEVDISGDCKVTYQAQQDKVVKIKALDTCKIERSGFTTANQVLGVSSKATSVT
TYKIEDSFVTAVLAEETRAFAMJFQQTIAGKTVSKQKLELKTTEAGPRMIPGKQVAGV
IKAVDSKYKAIPIVGQVLERVCKGCPSLAEHWKSIRKNLEPENLSKAEAVQSFLAFIQ
HLRTSRREEILQILKAEKKEVLPQLVDAVTSAQTPDSLEAILDFLDFKSDSSIILQER
FLYACGFATHPDEELLRALLSKFKGSFASNDIRESVMIIIGALVRKLCQNEGCKLKAV
VEAKKLILGGLEKPEKKEDTTMYLLALKNALLPEGIPLLLKYAEAGEGPVSHLATTVL
QRYDVSFITDEVKKTLNRIYHQNRKVHEKTVRTTAAAVILKNPSYMDVKNILLSIGEL
PKEMNKYMLTVVQDILHFEMPASKMIRRVLKEMAVHNYDRFSKSGSSSAYTGYVERSP
RAASTYSLDILYSGSGILRRSNLNIFQYIKGTELHGSQVVIEAQGLEGLIAATPDEGE
ENLDSYAGMSAILFDVQLRPVTFFNGYSDLMSKMLSASGDPVSVVKGLILLIDHSQDI
QLQSGLKANMEIQGGLAIDISGSMEFSLWYRESKTRVKNRVAVVITSDVTVDASFVKA
GLESRAETEAGLEFISTVQFSQYPFLVCMQMDKAEAPLRQFETKYERLSTGRGYVSRR
RKESLVAGCELPLHQQNSEMCNVVFPPQPESDNSGGWF

By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense MTP nucleic acid sequence, an anti-sense MTP nucleic acid sequence or both. Optionally, the siRNA is constructed such that a single transcript has both the sense and complementary antisense sequences from the target gene, e.g., a hairpin.

Binding of the siRNA to an MTP transcript in the target cell results in a reduction in MTP production by the cell. The length of the oligonucleotide is at least 10 nucleotides and may be as long as the naturally-occurring MTP transcript. Preferably, the oligonucleotide is 19-25 nucleotides in length. Most preferably, the oligonucleotide is less than 75, 50, 25 nucleotides in length. For example, the MTP siRNA includes the nucleotides at positions 480-580 of SEQ ID NO:4. MTP siRNA oligonucleotides which inhibit MTP expression in mammalian cells include oligonucleotides containing SEQ ID NO: 1 and 2. Exemplary MTP inhibitors include protease inhibitors, carboline compounds and compounds falling within Formula I or Formula II:
I. 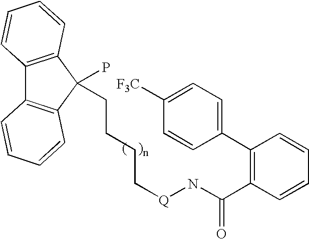
where n is zero or 1;
P is 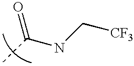
   or a 5- or 6-membered heterocycle selected from: 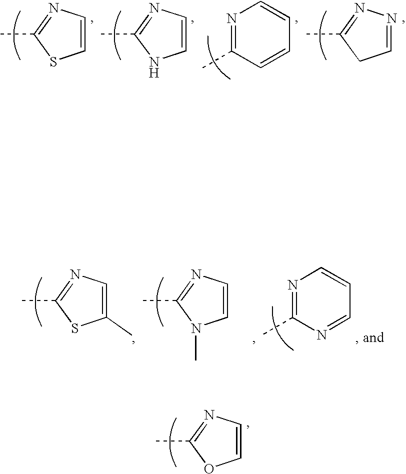
and Q is 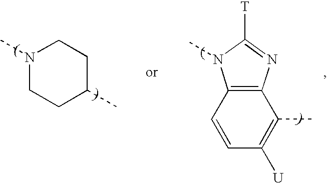
where T and U are, independently, hydrogen or lower alkyl. (See, Robl, J. A. et al, Journal of Medicinal Chemistry (2001), 44 (6): 851-6 and U.S. Pat. No. 6,281,228).
II. 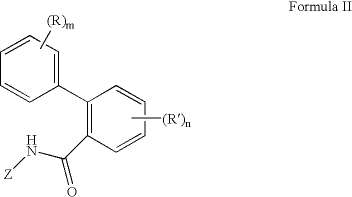
   where Z is selected from the group consisting of unsubstituted or substituted aryl, heteroaryl, alkyl, or heterocyclyl; m is 0, 1, 2, 3, 4 or 5; n is 0, 1, 2, 3, or 4; R is hydrogen, halogen, hydroxyl, amino, alkylamino, dialkylamino, carboxy, carboxyalkyl, nitro, cyano, lower alkoxy, or lower alkyl; and R′ is hydrogen, halogen, hydroxyl, lower alkoxy, or lower alkyl.

In one embodiment, Z is selected from the group consisting of:

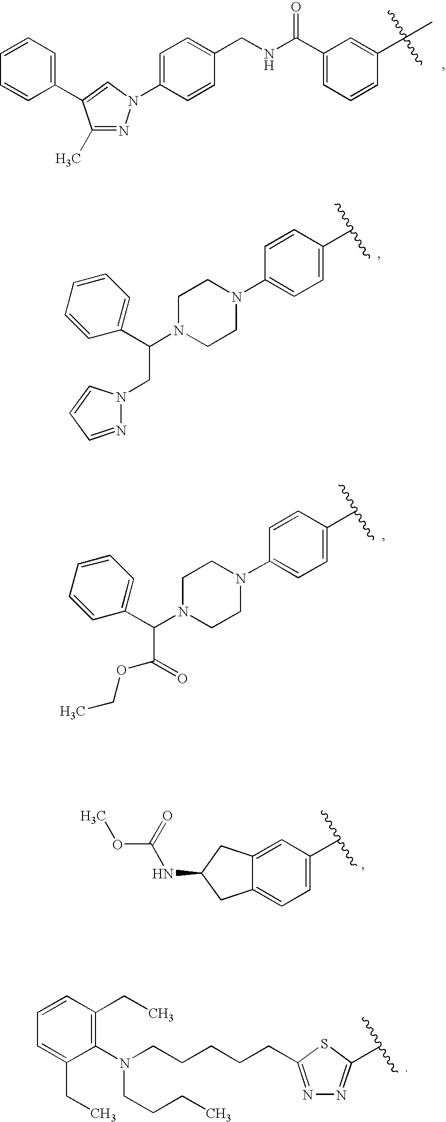

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. Unless the number of carbons is otherwise specified, “lower alkyl” as used herein means an alkyl group, as defined above, but having from one to ten carbons, more preferably from one to six carbon atoms in its backbone structure, and, if a cyclic alkyl, the term lower alkyl refers to those rings which have 5, 6, or 7 carbons in the ring structure. Alkyl groups optionally are substituted.

“Aryl” includes groups with aromaticity, including 5- and 6-membered “unconjugated”, or single-ring, aromatic groups that may include from zero to four heteroatoms, as well as “conjugated”, or multicyclic, systems with at least one aromatic ring. Examples of aryl groups include benzene, phenyl, pyrrole, furan, thiophene, thiazole, thiadiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, indole, benzofuran, purine, benzofuran, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions.

The term “alkoxy” includes substituted and unsubstituted alkyl groups covalently linked to an oxygen atom. Examples of alkoxy groups (or alkoxyl radicals) include methoxy, ethoxy, isopropyloxy, propoxy, butoxy, and pentoxy groups. The alkoxy groups can be substituted.

The terms “heterocyclyl” or “heterocyclic group” include closed ring structures, e.g., 3- to 10-, or 4- to 7-membered rings, which include one or more heteroatoms. “Heteroatom” includes atoms of any element other than carbon or hydrogen. Examples of heteroatoms include nitrogen, oxygen, sulfur and phosphorus.

Heterocyclyl groups can be saturated or unsaturated and include pyrrolidine, oxolane, thiolane, piperidine, piperazine, morpholine, lactones, lactams such as azetidinones and pyrrolidinones, sultams, and sultones. Heterocyclic groups such as pyrrole and furan can have aromatic character. They include fused ring structures such as quinoline and isoquinoline. Other examples of heterocyclic groups include pyridine and purine. The heterocyclic ring can be substituted at one or more positions.

The term “hydroxy” or “hydroxyl” includes groups with an —OH or —O⁻.

The term “halogen” includes fluorine, bromine, chlorine, iodine, etc.

As used herein, the term “amino” includes compounds where a nitrogen atom is covalently bonded to at least one carbon or heteroatom. The amino group can be substituted. “Alkylamino” includes groups wherein the nitrogen is bound to at least one additional alkyl group. “Dialkylamino” includes groups wherein the nitrogen atom is bound to at least two additional alkyl groups.

The term “carboxy” or “carbonyl” includes compounds and moieties which contain a carbon connected with a double bond to an oxygen atom. Examples of moieties containing a carbonyl include aldehydes, ketones, carboxylic acids, amides, esters, and anhydrides.

Protease inhibitors inhibit secretion of apolipoprotein B through inhibition of microsomal triglyceride transfer-protein activity. (Liang, J. et al, Nature Medicine (2001), 7 (12):1327-1331. Protease inhibitors include, for example, indinavir, ritonavir, nelfinavir and saquinavir. Other suitable protease inhibitors are known in the art.

Methods of Reducing Inflammation

Inflammation is inhibited by administering to tissue a MTP inhibitor. Tissues to be treated include a gastrointestinal tissue, e.g., intestinal tissue, a cardiac tissue, a pulmonary tissue, a dermal tissue, or a hepatic tissue. For example, the tissue is an epithelial tissue such as an intestinal epithelial tissue, pulmonary epithelial tissue, dermal tissue (i.e., skin), or liver epithelial tissue.

Inhibition of inflammation is characterized by a reduction of redness, pain and swelling of the treated tissue compared to a tissue that has not been contacted with a MTP inhibitor. Tissues are directly contacted with an inhibitor. Alternatively, the inhibitor is administered systemically. MTP inhibitors are administered in an amount sufficient to decrease (e.g., inhibit) inflammatory cytokine production. An inflammatory cytokine is a cytokine that modulates, e.g., induces or reduces an inflammatory response. An inflammatory response is evaluated by morphologically by observing tissue damage, localized redness, and swelling of the affected area. Alternatively, an inflammatory response is evaluated by measuring c-reactive protein, or IL-1 in the tissue or in the serum or plasma. An inflammatory cytokine is a proinflammatory cytokine. For example the inflammatory cytokine is, TNF alpha, interferon (e.g., alpha, beta or gamma), or interleukin (e.g., IL-1, IL-6, IL-10, IL-12, IL-14, IL-18). Cytokines are detected for example in the serum, plasma or the tissue. Cytokine production is measured by methods know in the art. For example, cytokine production is determined using an immunoassay specific for a Th1-specific or Th2-specific cytokine. A decrease in production of the cytokine in the presence of the compound compared to the level in the absence of the compound indicates a decrease in cytokine production. A decrease in white blood count also indicates a decrease in inflammation.

Efficaciousness of treatment is determined in association with any known method for diagnosing or treating the particular inflammatory disorder. Alleviation of one or more symptoms of the inflammatory disorder indicates that the compound confers a clinical benefit.

Alternatively, the cell is contacted with a MTP inhibitor in an amount sufficient to decrease T-cell activation, decrease antigen presentation or decrease expression of CD1d. T cells include T cytotoxic cells, T helper cells (e.g., Th1 and Th2) and natural killer T-Cells. The T-cell is a CD1d restricted T-cell. The T-cell express the natural killer receptor or an invariant T-cell receptor. T-cell activation is defined by an increase in calcium mediated intracellular cGMP or an increase in cell surface receptors for IL-2. For example, a decrease of T-cell activation is characterized by a decrease of calcium mediated intracellular cGMP and or IL-2 receptors in the presence of the compound compared to a the absence of the compound. Intracellular cGMP is measured for example, by a competitive immunoassay or scintillation proximity assay using commercially available test kits. Cell surface IL-2 receptors are measured for example, by determining binding to an IL-2 receptor antibody such as the PC61 antibody.

Antigen presentation the expression antigen on surface of a cell in a form recognizable by lymphocytes. Antigen presentation is determined by methods known in the art such as measuring IFN gamma production or IL-2 production. For example, an decrease of IFN gamma or IL-2 production in the presence of the MTP inhibitor as compared to the absence of the MTP inhibitor indicates a decrease in antigen presentation. IFN gamma or IL-2 production is measured for example, by binding to an ILN gamma or IL-2 antibody. A decrease in CD1d expression is defined by a decrease in cell surface expression. A decrease in CD1d cell surface expression is measured for example, by determining binding to a CD1d antibody.

Target cells includes those which express microsomal triglyceride transfer protein or which are induced to express MTP upon exposure to an inflammation trigger, e.g., infection, tissue damage, or exposure to an allergen. The target cells express CD1d or any CD1 molecule. The cell is an immune cell such as an antigen presenting cell. The immune cell is for example as a B-cell, a monocyte, a macrophage, or a dendritic cell. The cell is a heart cell, a kidney cell, a brain cell, a yolk sac cell, a liver cell (i.e. hepatocyte), an epithelial cell or an intestinal cell. Preferably, the cell is an epithelial cell of the large or small intestine or the lung.

The methods are useful to alleviate the symptoms of a variety of inflammatory disorders. The inflammatory disorder is acute or chronic. Inflammatory disorders include cardiovascular inflammation, gastrointestinal inflammation, hepatic inflammatory disorders, pulmonary inflammation, kidney inflammation, ocular inflammation, pancreatic inflammation, genitourinary inflammation, autoimmune disease (e.g., diabetes, systemic lupus erythematosus, dermatomyositis, polymyositis, inflammatory neuropathies (Guillain Barre, inflammatory polyneuropathies), vasculitis (Wegener's granulomatosus, polyarteritis nodosa), polymyalgia rheumatica, temporal arteritis, Sjogren's syndrome, Bechet's disease, Churg-Strauss syndrome, Takayasu's arteritis), neuroinflammatory disorders (e.g., multiple sclerosis, allergy (e.g., allergic rhinitis/sinusitis, skin allergies and disorders (e.g., urticaria/hives, angioedema, atopic dermatitis, contact dermatitis, psoriasis), food allergies, drug allergies, insect allergies, mastocytosis), musculoskeletal inflammation (e.g., arthritis, myositis, osteoarthritis, rheumatoid arthritis, spondyloarthropathies), dermatologic disorders, endocrinologic disorders (e.g., thyroiditis) infection (e.g., bacterial or viral infections that depend on CD1d presentation such as Borrelia burgdorferi, Cryptococcus neoformans, Plasmodium falciparum, Trypanosoma cruzi, Leishmania major, influenza, or viral hepatitis); oral inflammatory disorders (i.e., perodontis, gingivitis or somatitis); and transplantation (e.g., allograft or xenograft rejection or maternal-fetal tolerance).

The methods described herein lead to a reduction in the severity or the alleviation of one or more symptoms of an inflammatory disorder such as those described herein. Inflammatory disorders are diagnosed and or monitored, typically by a physician using standard methodologies

Gastrointestinal Inflammatory Disorders

Gastrointestinal inflammatory disorders include for example, inflammatory bowel disease, Crohn's Disease, colitis (i.e., ulcerative, ileitis or proctitis).

Ulcerative colitis is an inflammatory bowel disease that causes inflammation and sores, called ulcers, in the top layers of the lining of the large intestine. The inflammation usually occurs in the rectum and lower part of the colon, but it can affect the entire colon. Ulcerative colitis rarely affects the small intestine except for the lower section, called the ileum. Ulcerative colitis occurs most often in people ages 15 to 40, although children and older people develop the disease. Ulcerative colitis affects men and women equally and appears to run in families. Crohn's Disease causes inflammation deeper within the intestinal wall. Crohn's disease usually occurs in the small intestine, but it can also occur in the mouth, esophagus, stomach, duodenum, large intestine, appendix, and anus.

Symptoms of gastrointestinal inflammatory disorder are abdominal pain and bloody diarrhea. Other symptoms include fatigue, weight loss, loss of appetite, rectal bleeding and loss of body fluids and nutrients. Gastrointestinal inflammation can also cause problems such as arthritis, inflammation of the eye, liver disease (fatty liver, hepatitis, cirrhosis, and primary sclerosing cholangitis), osteoporosis, skin rashes, anemia, and kidney stones.

Gastrointestinal inflammation is diagnosed using a blood tests to check for anemia, which can indicate bleeding in the colon or rectum. In addition, a stool sample, can be taken to determine is there is bleeding or infection in the colon or rectum. Alternatively, a colonoscopy is performed to detect inflammation, bleeding, or ulcers on the colon wall.

Hepatic Inflammatory Disorders

Hepatic inflammatory disorders include for example, hepatitis such viral hepatitis, bacterial hepatitis, autoimmune hepatitis, drug induced hepatitis or alcoholic hepatitis. The incidence and severity of hepatitis vary depending on many factors, including the cause of the liver damage and any underlying illnesses in a patient. Common risk factors include intravenous drug use, Tylenol overdose (the dose needed to cause damage is quite close to the effective dose so be sure to be careful to take Tylenol only as directed), risky sexual behaviors, ingestion of contaminated foods, and alcohol use.

Symptoms of hepatitis include dark urine, loss of appetite fatigue, jaundice, abdominal pain, black stool. Hepatitis is diagnosed by physical exam, liver function test, autoimmune marker and serology.

Pulmonary Inflammatory Disorders

Pulmonary inflammatory disorders include for example, sinusitis acute respiratory distress syndrome, asthma, bronchopulmonary dysplasia (BPD), emphysema, interstitial lung diseases, lung injury, and pulmonary hypertension.

Asthma is a chronic lung condition that can develop at any age. It is most common in childhood and occurs in approximately 7-10% of the pediatric population. Asthma affects twice as many boys as girls in childhood; more girls than boys develop asthma as teenagers, and in adulthood, the ratio becomes 1:1 males to females. Symptoms of asthma include shortness of breath, wheezing, constriction of the chest muscles, coughing, sputum production, excess rapid breathing/gasping, rapid heart rate and exhaustion. Asthma is diagnosed by physical examination, i.e. listening to the lungs with a stethoscope; examination of nasal passages, chest x-ray, blood tests or spirometry.

Cardiac Disorders

Cardiac inflammatory disorders include for example pericarditis, endocarditis, mycocarditis and atherosclerosis. Cardiac inflammation also includes an inflammation that results from an acute cardiac event such as a myocardial infarction. Cardiac inflammation is distinguished from other cardiac disorders in that inflammation is typically acute while other disorder such atherosclerosis are chronic. Atherosclerosis results in the build up of deposits of fatty substances, cholesterol, cellular waste products, calcium and in the inner lining of an artery (i.e., plaque) and has a significant inflammatory component. In contrast, cardiac inflammation affects the muscle tissue of the heart.

Pericarditis, is inflammation of the pericardium and is characterized by chest pain. Patients who have suffered a myocardial infarction often develop pericarditis over subsequent days or weeks. Pericarditis is diagnosed by elevated ST segments on an electrocardiogram.

Endocarditis is the inflammation of the endocardium and causes a wide variety of symptoms, particularly in the earlier stages of infection. Symptoms include fevers, chills, fatigue, weight loss, muscle aches, and sweating. Endocarditis is diagnoses by the presence of a heart murmur or an echocardiogram.

Myocarditis is the inflammation of the heart muscle. The symptoms of myocarditis include fever, chest pain, abnormal heat beats, fatique and shortness of breath. Myocarditis is typically diagnosed by a endomyocardial biopsy.

Method of Increasing Immunosurveillance

MTP inhibitors block or reduce immuno-evasive mechanisms of neoplastic cells. The success of immunotherapy against cancer depends on how efficiently tumor antigens are presented to antigen specific T-cells and how efficiently these T-cells are activated to eradicate tumor cells. May sub clinical tumors expressing immunogenic tumor antigens spontaneously regress, only those that escape immunosurveillance reach the stage at which they are detected clinically. CD1-deficient mice are resistant to tumor recurrence, indicating that CD1 restricted T-cells are necessary for the down regulation in immunosurveillance.

Immunosurveillance is increased by contacting a cell or administering to a subject a MTP inhibitor. An increase in immunosurveillance is characterized by a reduction in tumor recurrence. Cells are directly contacted with an inhibitor. Alternatively, the inhibitor is administered systemically. MTP inhibitors are administered in an amount sufficient to decrease (e.g., inhibit), cytokine production such as IL-13 production and or NKT cell activation compared to a cell that has not been contacted with the inhibitor. IL-13 is detected for example in the serum, plasma or the tissue. Il-13 is measured by methods know in the art. For example, cytokine production is determined using an immunoassay specific for IL-13. A decrease in production of IL-13 in the presence of the MTP inhibitor compared to the level in the absence of the compound indicates a mechanism of immune evasion by tumor cells. NKT cell activation is determined by methods described herein or methods know in the art.

The subject is suffering from or at risk of developing a tumor. A subject suffering from or at risk of developing tumor is identified by methods known in the art, e.g., gross examination of tissue, e.g. biopsy, x-rays, CT scans, MRIs or detection of a tumor associated antigen in tissue or blood. Symptoms cancer include a lump or thickening in the breast or testicles; a change in a wart or mole; a skin sore or a persistent sore throat that doesn't heal; a change in bowel or bladder habits; a persistent cough or coughing blood; constant indigestion or trouble swallowing; unusual bleeding or vaginal discharge; and chronic fatigue.

The methods are useful to alleviate the symptoms of a variety of neoplastic disorders Neoplastic disorders include for example malignant transformations of parenchymal tissues such as the colon, lung, breast, kidneys, bones, head & neck, musculoskeletal systems, liver, endocrine organs, genitourinary system including ovaries and testes and skin or hematopoietic system including leukemias and lymphomas. These conditions affect individuals of all ages and are diagnosed by virtue of the detection of abormal growths of cells in the form of masses or abnormal accumulations of cells in organs or the symptoms that they elicit that are either nonspecific (e.g. fevers, weight loss or loss of apetite) or specific for the particular tumor. In these conditions, uncontrolled proliferation of specific cell types occurs that eludes the abilities of the immune system to eradicate and control the abnormal growth. In many cases, the tumors actively evade the response of the immune system by a variety of different strategies. These include suppression of the immune system by inducing, for example, the secretion of proteins or cytokines that are inhibitory to an immune response. Blocking these immune evasive mechanisms would be beneficial to the human with a neoplastic condition.

Therapeutic Administration

The invention includes administering to a subject a composition comprising a compound that decreases MTP expression or activity (referred to herein as an “MTP inhibitor” or “therapeutic compound”).

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-inflammatory agents or therapeutic agents for treating, preventing or alleviating a symptom of a particular inflammatory disorder. A therapeutic regimen is carried out by identifying a mammal, e.g., a human patient suffering from (or at risk of developing) an inflammatory disorder, using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The compound is administered prophylactically, or after the detection of an inflammatory event such as an asthma attack or an allergic reaction. The compound is optionally formulated as a component of a cocktail of therapeutic drugs to treat inflammatory disorders. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, MTP inhibitor is formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

MTP inhibitor compounds are effective upon direct contact of the compound with the affected tissue. Accordingly, the compound is administered topically. For example, to treat contact dermatitis the compound is applied to the area of skin affected. Alternatively, MTP inhibitors are administered systemically. Additionally, compounds are administered by implanting (either directly into an organ such as the intestine, or liver or subcutaneously) a solid or resorbable matrix which slowly releases the compound into adjacent and surrounding tissues of the subject.

For example, for the treatment of gastrointestinal inflammatory disorders, the compound is systemically administered or locally administered directly into gastric tissue. The systemic administration compound is administered intravenously, rectally or orally. For local administration, a compound-impregnated wafer or resorbable sponge is placed in direct contact with gastric tissue. The compound or mixture of compounds is slowly released in vivo by diffusion of the drug from the wafer and erosion of the polymer matrix.

Inflammation of the liver (i.e., hepatitis) is treated for example by infusing into the liver vasculature a solution containing the compound. Intraperitoneal infusion or lavage is useful to reduce generalized intraperitioneal inflammation of prevent inflammation following a surgical event.

For the treatment of neurological inflammation the compound is administered intravenously or intrathecally (i.e., by direct infusion into the cerebrospinal fluid). For local administration, a compound-impregnated wafer or resorbable sponge is placed in direct contact with CNS tissue. The compound or mixture of compounds is slowly released in vivo by diffusion of the drug from the wafer and erosion of the polymer matrix. Alternatively, the compound is infused into the brain or cerebrospinal fluid using known methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (e.g., an assembly described in U.S. Pat. No. 5,954,687) provides a fluid flow path suitable for the transfer of fluids to or from selected location at, near or within the brain to allow administration of the drug over a period of time.

For treatment of cardiac inflammation, the compound is delivered for example to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen. Any variety of coronary catheter, or a perfusion catheter, is used to administer the compound. Alternatively, the compound is coated or impregnated on a stent that is placed in a coronary vessel.

Pulmonary inflammation is treated for example by administering the compound by inhalation. The compounds are delivered in the form of an aerosol spray from pressured container or dispenser which contains a suitable propellant, e.g., a gas such as carbon dioxide, or a nebulizer.

Evaluation of Anti-Inflammatory Activity

Anti-inflammatory activity of a compound is identified by incubating cell with a compound and measuring inflammatory activity. A decrease in inflammation in the presence of the compound compared to the level in the absence of the compound indicates that the compound inhibits inflammation. Inflammatory activity is measured by detecting the production of cytokines such as IL-1. An increase in production of IL-1 in the presence of the compound compared to the amount detected in the absence of the compound indicates that the compound inhibits inflammation. A decrease in production of IL-1 in the presence of the compound compared to the amount detected in the absence of the compound indicates that the compound promotes inflammation. Inflammatory activity is also measured by detecting the amount of c-reactive protein or determining the erythrocyte sedimentation rate (ESR).

The following methods and reagents were used to generate the data described herein

Cells

The mouse (MODE-K) small IEC line has been previously described (van de Waal, Y. et al. Gastroenterology. 124, 1420-1431, (2003)). The murine Vα14/Jα281 invariant TCR-positive T cell hybridoma, DN32.D3, was kindly provided by Dr. Albert Bendelac (University of Chicago, Chicago, Ill.) (Bendelac, A. et al. Science. 268, 863-69 (1995)). DN32.D3 and MODE-K cells or primary murine hepatocytes were cultured in Dulbecco's modification of Eagle's medium (DMEM) (Gibco™ Invitrogen, Carlsbad, Calif.) supplemented with 10% FBS (Sigma-Aldrich, St. Louis, Mo.), 100 U/ml penicillin, 100 μg/ml streptomycin (Bio Whittaker, Walkersville, Md.), 10 mM HEPES (Gibco Invitrogen), and 1% non-essential amino acid (Mediatech, Herndon, Va.), as complete DMEM.

Cells were maintained in R10 (RPMI 1640 medium with 2 mM L-glutamine; 10% fetal bovine serum) unless otherwise indicated. MODE-K, a mouse IEC line, has been previously described. DN32.D3 (called DN32 here), a mouse Va14Ja18 invariant TCR-positive T-cell hybridoma, was provided by A. Bendelac (University of Chicago). The autoreactive mouse Vα14Jα18 invariant TCR-positive hybridoma 24.8 was provided by S. Behar (Brigham and Women's Hospital). RMAS is a TAP-deficient mouse T cell lymphoma; RAW is an Abelson virus transformed mouse macrophage cell line (ATCC# TIB-71). Jurkat is a human T lymphocyte line; U937 is a human histocytic lymphoma cell line. C1R, a human B cell line which lacks expression of MHC class I, was transfected with human CD1d as previously described to generate C1Rd.

Animals

Conditional MTPp gene deficient mice (MTP^{flox/flox}) were bred with Mx1 promoter-driven Cre recombinase transgenic mice as previously described to generate MTPmx1 (previously referred to as Mttp^{flox/flox}/Mx1-Cre) mice (Raabe, M. et al. J. Clin. Invest. 103, 1287-1298 (1999)). Wild type (WT) C57BL/6J (B6) mice were purchased from Charles River Breeding Laboratories (Wilmington, Mass.). Mice were maintained under specific pathogen free conditions at the Animal Facilities of Harvard Medical School, Boston, Mass. All animal experimentations were performed in accordance with institutional guidelines and the Review Board of Harvard Medical School, which has granted permission for this study.

Assessment of Hepatic and Plasma Lipids After MTPp Deletion

Female 6-8 week-old of both MTP^{pflox/flox}Mx1Cre mice and WT B6 mice that had been injected intraperitoneally with either PBS as control or 500 μg polyinosinic-polycytidylic ribonucleic acid (pIpC, Sigma, St. Louis, Mo.) in PBS every other day for a total of four doses were sacrificed one or 10 days after the last pIpC injection at which time sera was obtained from peripheral blood. The serum was subjected to total cholesterol and triglyceride assays at the clinical laboratory of Brigham and Women's Hospital (Boston, Mass.).

Fresh liver samples that were collected from mice before pIpC injection or one day and 10 days after injection and immediately placed in OCT compound (Sakura Finetek USA, Inc., Torrance, Calif.) on dry ice and 5 μm thick cryosections prepared. Sections were air dried and fixed in 10% formalin (Fisher, Pittsburgh, Pa.) for 30 min followed by administration of oil-red-O by a standard protocol. Briefly, samples were washed with distilled water and rinsed twice for 5 min in 100% propylene glycol (Fisher). The samples were then stained with 7 mg/ml oil-red-O (Sigma) dissolved in propylene glycol with agitation and subsequently soaked in 85% (v/v with distilled water) propylene glycol for 3 min. After rinsing with distilled water, nuclear staining was performed with hematoxylin (VWR, West Chester, Pa.).

Cell Isolations and Antigen Presentation Assays

Fresh livers and colons were collected from MTP^{flox/flox}Mx1Cre mice treated with either PBS or pIpC. Livers were crushed on 70 μm cell strainers (BD Biosciences, San Jose, Calif.) and total hepatocytes were further fractionated on a 30% Percoll (Amersham Biosciences) gradient by centrifugation at 2000 rpm for 20 min. Primary hepatocytes were isolated from the surface fraction and antigen presentation assays performed as previously described. Briefly, 1×10⁵ primary hepatocytes in 100 μl DMEM were loaded overnight with either vehicle or 100 ng/ml αGalCer in flat bottom 96-well plates. The plates were subsequently washed twice with PBS preceding the addition of 5×10⁴ DN32.D3 cells. Culture supernatants were harvested after 24 h and subjected to determination of murine IL-2 production by ELISA (OptEIA, BD Pharmingen, San Diego, Calif.) according to the manufacturer's instructions.

Colonic specimens were washed with HBSS, cut in 3 mm pieces and incubated twice in HBSS containing 5 mM EDTA (Sigma) and 1 mM DTT (Sigma) in a shaking incubator at 37° C. for 20 min. Following the collection of supernatant, cells were further fractionated on a 30% Percoll gradient by centrifugation at 2000 rpm for 20 min. Primary colonic epithelial cells were isolated from the surface fraction.

APCs were incubated with 100 ng/mL α-galcer (kindly provided by Dr. H. Ploegh) for 3 hours, washed three times in PBS and aliquoted into a 96 well plate at 10⁶ cells/mL. DN32 cells were added at 1:1 E:T ratio unless otherwise indicated. CD4⁺ cells were isolated from spleens of OT-II transgenic mice using positive selection on CD4 magnetic beads (Miltenyi Biotec) and incubated with APCs and 100 μg/mL ovalbumin. The 24.8 NKT cell line was incubated with APCs in the absence of added antigen at E:T=2:1 unless otherwise indicated. Mouse IL-2 production was assessed by ELISA (OptEIA, BD PharMingen) of 24 or 40 hour culture supernatants. APCs were treated with BMS212122 (10 uM dissolved in DMSO; kindly provided by Bristol Myers Squibb, New Jersey) as indicated and washed prior to addition of T or NKT cells. Human NKT cell lines generated from healthy donor leukopaks (>90% pure) were incubated with 1 ng/mL PMA and C1Rd or C1R mock transfected cells in the presence of BMS 197636 (kindly provided by Bristol Myers Squibb) or DMSO control at the indicated concentrations.

The expression levels of MTP protein in primary hepatocytes and colonic epithelial cells were assessed by RT-PCR (see below) and Western blotting by a standard protocol. Briefly, 20 μg cell lysates were applied to 6% SDS-PAGE and then transferred to a nitrocellulose membrane. Following the blocking with 5% skim milk, the membrane was incubated with specific antibodies and the signals generated were detected by ECL Western blotting analysis system (Amersham Biosciences). The specific antibodies used included either anti-CD1d monoclonal antibody, IBI (BD Pharmingen), anti-MTP antibody (BD Biosciences), goat anti-mouse IgG2α-HRP (Southern Biotechnology Associates, Inc., Birmingham, Ala.), rat IgG2b antibody (BD Pharmingen) or mouse IgG2a (BD Pharmingen).

Reverse Transcriptase-Polymerase Chain Reaction Amplification (RT-PCR)

Total cellular RNA was extracted using Trizol (Invitrogen, Carlsbad, Calif.) following the manufacture's instructions. and cDNA was synthesized with Powerscript reverse transcriptase (Clontech Laboratories).

100 ng RNA was subjected to reverse-transcription using the Advantage RT-for-PCR Kit (Clontech Laboratories, Palo Alto, Calif.). The sense and antisense primers for murine MTPp were 5′-GGACTTTTTGGATTTCAAAAGTGAC-3′ (SEQ ID NO:7) and 5′-GGAGAAACGGTCATAATTGTG-3′ (SEQ ID NO:8). The conditions for the PCR were as follows: after initial denaturation at 95° C. for 5 min, the thermocycler (MJ Research, Watertown, Mass.) was programmed for 35 cycles: 1 min at 95° C., 1 min at 55° C. and 2 min at 72° C. The reaction was concluded with a final extension step at 72° C. for 7 min. 1 μl of each PCR reaction mixture was separated and visualized with 1.5% agarose gel electrophoresis containing 0.01% ethidium bromide. Optical density of the cDNA bands were determined by a computerized image-analysis system and normalized to the RT-PCR products of α-actin with the following primer pair: 5′-GTGGGCCGCTCTAGGCACCAA-3′ (SEQ ID NO:9) and 5′-CTCTTTGATGTCACGCACGATTTC-3′ (SEQ ID NO: 10).

Mttp transcripts were amplified by PCR using 5 μL of cDNA per reaction and primers 5′-GGACTTTTTGGATTTCAAAAGTGAC-3′ (SEQ ID NO:11) and 5′-GGAGAAACGGTCATAATTGTG-3′ (SEQ ID NO:12) which amplify both mouse (696 bp) and human (699 bp) transcripts. PCR reactions were heated at 94° C. for 3 minutes followed by 35 cycles of 94° C. for 40 sec., 53° C. for 90 sec., 72° C. for 60 sec. Samples were loaded onto a 1.2% agarose gel and visualized by ethidium bromide staining. The volumes of the PCR products loaded were normalized to beta-actin transcripts amplified using 2 μL cDNA and primers 5′-ATCTGGCACCACACCTTCTACATTGAGCTGCG-3′ (SEQ ID NO:13) and 5′-CGTCATACTCCTGCTTGCTGATCCACATCTGC-3′ (SEQ ID NO:14).

Immunohistochemistry and Confocal Microscopy

MTP^{flox/flox}Mx1Cre mice that had been treated with either vehicle or pIpC were sacrificed and fresh liver samples immediately placed in OCT compound (Sakura Finetek USA) on dry ice and 5 μm cryosections prepared. Sections were air dried and fixed in cold acetone for 30 min. Samples were rehydrated in PBS for 5 min and blocked in PBS with 10% goat serum (Zymed Laboratories, Inc South San Francisco, Calif.) for 20 min. The sections were incubated with rat anti-mouse CD1d monoclonal antibody, 1B1, or rat anti-mouse CEACAM1 antibody (clone AgB10) (kindly provided by Dr. Nicole Beauchemin, McGill University, Montreal, Canada), followed by Alexa⁴⁸⁸ conjugated goat anti-rat IgG secondary antibodies and rhodamine conjugated phalloidin (Molecular Probe, Eugene, Oreg.) for 30 min at RT. After washing three times in PBS for 5 min, nuclei were stained with TO-PRO-3 (Molecular probe) and tissues mounted and preserved with Prolong Antifade reagent (Molecular probe). All images were collected using a MRC1024 laser scanning confocal system (Bio-Rad Laboratories, Hercules, Calif.) using the same laser power, gain, and pinhole size for the respective channels.

αGalCer-Induced Hepatitis

αGalCer was kindly provided by Dr. Michael Brenner (Brigham and Women's Hospital, Boston, Mass.). To generate αGalCer-induced hepatitis, 6 week-old female WT B6 or MTP^{flox/flox}Mx1Cre mice were injected intraperitoneally with PBS or 500 μg pIpC at days 1, 3, 5 and 7 and injected intraperitoneally with 2 μg/mouse of αGalCer on either day 8 or day 18. Mice were sacrificed and sera from peripheral blood subjected to aminoleucine transferase (ALT) or aminoaspartate transferase (AST) assays. The kinetic quantitative determination of ALT in serum was performed by ALT/SGPT LIQUI-UV (Stanbio Laboratory, Boeme, Tex.) according to the manufacture's instructions. Fresh liver samples were collected for macroscopic and microscopic inspection. For microscopic examination, liver tissues were immediately fixed in 10% buffered formalin phosphate and embedded in paraffin, cut into sections and stained with hematoxylin-eosin.

Mttp Gene Silencing by Means of Small Interference RNA

To selectively silence MTPp gene expression in the murine IEC line, MODE-K, a specific small interference (si) RNA approach was developed. The siRNA duplexes were generated and consisted of a sense strand (5′-AAGCUCUGGAACUACCAACGAdTdT-3′ SEQ ID NO:15)) and an anti-sense strand (5′-UCGUUGGUAGUUCCAGAGCUUdTdT-3′ SEQ ID NO:16) (Xeragon Inc. Germantown, Md.). 3 μg siRNA was used to transfect 5×10⁵ cells using the TransMessenger Transfect kit (Qiagen Inc., Valencia, Calif.) following the manufacturer's instructions. To confirm the post-transcriptional gene silencing effect of MTPp-specific siRNA, the transfected cells were harvested 48 h after transfection. Total cellular RNA was then extracted and subjected to RT-PCR analysis as described above.

U937 cells in ATCC complete media (RPMI 1640 medium with 2 mM L-glutamine adjusted to contain 1.5 g/L sodium bicarbonate, 4.5 g/L glucose, 10 mM HEPES, and 1.0 mM sodium pyruvate, 90%; fetal bovine serum, 10%) were aliquoted into a 6 well plate and simultaneously transfected with two human mttp specific siRNA oligos at 25 nM each (target sequences: NNUUAUGACCGUUUCUCCAGG (SEQ ID NO:17); AAGCUCACGUACUCCACUGAA (SEQ ID NO:18)) or transfected with mock siRNA (specific for mouse but not human mttp) at 50 nM. Oligos were complexed with siPORT amine (Ambion) as per the manufacturer's instructions and added to U937 cells for a final culture volume of 2 mL. Fresh media was added 24 hours later. Cells were harvested 72 hours later, incubated with 100 ng/mL α-galactosylceramide (α-galcer) or vehicle, washed and cocultured with DN32 cells (E:T=1:1) for 24 hours. Culture supernatants were collected and assayed for IL-2 by sandwich ELISA. A portion of the 72 hour silenced cells were used for RNA extraction, and mttp transcript knockdown was measured by RT-PCR.

Hapten-Induced Colitis

The hapten-induced colitis model using oxazolone (4-ethoxymethylene-2-phenyl-2-oxazolin-5-one) as the hapten was established in WT B6 or MTP^{flox/flox}Mx1Cre mice as previously described. Briefly, 6 week-old female mice were injected intraperitoneally with vehicle or 500 μg pIpC at days 1, 3, 5 and 7 followed by application of 200 μl of 3% oxazolone (Sigma-Aldrich, St. Louis, Mo.) in 100% ethanol on the abdomen at day 8. On day 14, mice were anesthetized with tribromo-ethanol (Sigma-Aldrich) and 150 μl of 1% oxazolone in 50% ethanol administered per rectum via a 3.5F catheter. Wasting was monitored until day 18 at which time the mice were sacrificed and tissue samples collected for histological examination. Colonic tissue specimens for histological assessment of colitis were fixed in 10% buffered formalin phosphate and embedded in paraffin, cut into 5 micron thick sections and stained with hematoxylin-eosin. The stained paraffin sections were evaluated by for the following parameters of colitis on a scale of 0 to 3 according to known methods: hypervascularity, mononuclear cell infiltration, crypt hyperplasia, epithelial injury or ulceration, polymorphonuclear cell infiltration or crypt abscesses.

Microsome Preparation and Western Blotting.

C57BL/6 mice were injected with flt3L secreting melanoma provided by G. Dranoff (Dana Farber Cancer Institute), and splenocytes were harvested 1-2 weeks later. To prepare microsomes, splenocytes were resuspended in isotonic buffer (250 mM sucrose, 5 mM imidazole, 1 mM EDTA, complete protease inhibitors) and homogenized on ice for 50 strokes using a glass manual homogenizer. The cell lysates were centrifuged at 250 g to remove nuclei and unbroken cells followed by centrifugation at 10,000 g for 30 minutes to remove mitochondria. Microsomes were pelleted at 100,000 g for 2 hours, resuspended in isotonic buffer and stored at 4 degrees. Separation by SDS-PAGE was done by standard methods followed by immunoblotting with mouse antibody to mouse MTP (IgG2a; BD Biosciences).

Triglyceride and PE Transfer Assays.

The triglyceride transfer activity of MTP was measured using donor phospholipids vesicles (Chylos Inc.) as described. PE transfer was done by overnight coating of a microtiter plate with 2 ug per well recombinant murine CD1d purified by baculovirus expression system. Wells were washed with PBS, incubated with PBS containing 0.5% isopropanol for 2 hours at 37° C., and washed again with PBS. MTP purified from rat liver (>95% pure by SDS-PAGE) and PE vesicles containing 6:1 PE:NBD-PE were resuspended in transfer buffer (1 mM Tris-HCl, pH 7.4, 0.2 mM EDTA, 15 mM NaCl, 0.1% fatty-acid free BSA from Sigma), added to the wells and incubated at 37° C. for 2 hours. Wells were then washed three times with PBS. 100 μL isopropanol was added to each well for 60 seconds and then transferred to a black microtiter plate (Thermo Labsystems, Franklin, Mass.). Fluorescence was read with a fluorescence plate reader (7620 Microplate Fluorimeter; Cambridge Technology, Watertown, Mass.) using 460 nm excitation and 530 nm emission wavelengths.

Dendritic Cell Cultures.

Bone marrow was extracted from the femurs of C57BL6 or MTPmx1cre mice, washed once in PBS and resuspended in R10 supplemented with culture supernatant from murine GM-CSF transfected cells for a final concentration of 200 U/mL GM-CSF. BMDCs were then cultured in bacteriological plates for 12-14 days as described. Differentiated cells were harvested by gentle pipetting, washed in PBS, and analyzed by flow cytometry for surface expression of CD11c and I-A^{b}. Human monocytes were harvested from the peripheral blood of healthy volunteers by positive selection on CD14 magnetic beads (Miltenyi Biotec) and cultured in R10 medium at 10⁶ cells per mL supplemented with 200 U/mL recombinant hIL-4 and 300 U/mL recombinant hGM-CSF (PeproTech, Rocky Hill, N.J.). After 5 days cells were dislodged by gentle pipetting, analyzed by flow cytometry and assayed for antigen presentation.

Flow Cytometry.

Mouse cells were stained using the following antibodies: FITC-conjugated α-CD1d (1B1, PharMingen); PE-conjugated α-CD11c (HL3, PharMingen); FITC-conjugated α-1-A^{d} (AMS-32.1, PharMingen); PE-conjugated α-CD69 (H1.2F3, PharMingen); PE-conjugated α-B220 (RA3-6B2, PharMingen). Human cells were stained using: PE-conjugated α-CD83 (550634, PharMingen); FITC-conjugated α-HLA-A,B,C (557348, PharMingen); α-HLA DR,DP,DQ (32381A, PharMingen); α-CD1d (42.1 or 51.1.3); α-mouse IgG+IgM (AMI1708, Biosource, Camarillo, Calif.). Staining was performed in the presence of Via-Probe (PharMingen) and analyzed with a FACSort flow cytometer (Becton Dickinson).

Statistical Analysis

Data are expressed as the mean±SEM and statistical significance determined by the Student's t-test. P values <0.05 were considered significant.

### EXAMPLE 1

### MTPp Gene Deletion Results in Redistribution of CD1d Expression in Hepatocytes

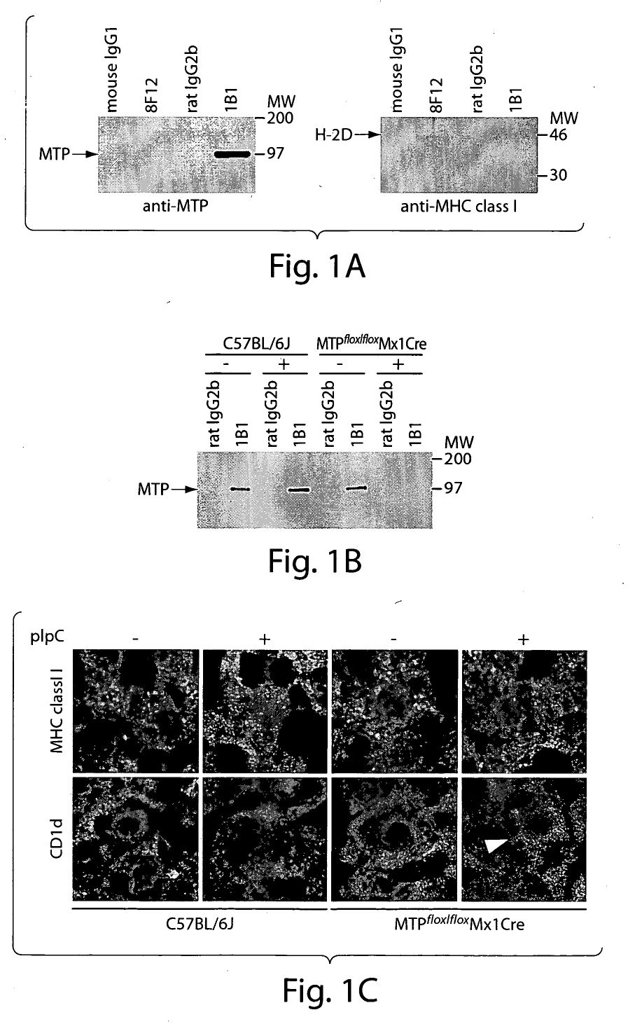

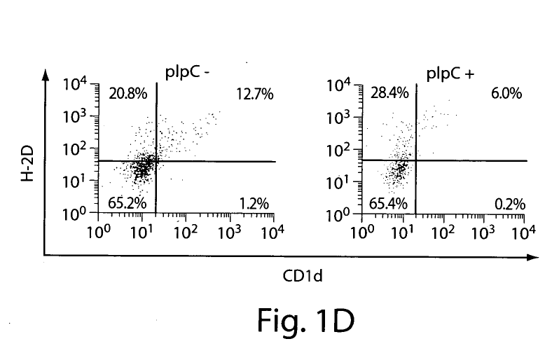

CD1d and MTP were found to co-associate with each other in hepatocytes. As shown in FIG. 1A, when protein lysates of hepatocytes from C57BL/6J mice were immunoprecipitated with a CD1d-specific monoclonal antibody and the immunoprecipitates resolved by SDS-PAGE followed by Western blotting, a specific band of 97-kDa consistent with MTP was detected.

To further substantiate a direct biochemical relationship between CD1d and MTP, the effects on CD1d expression and function was examined in a conditional MTP-deficient mouse model which contains a “floxed” MTPp gene (MTPp^{flox/flox}) that had been intercrossed with mice expressing Mx1 promoter controlled, Cre-recombinase (Raabe, M. et al. J. Clin. Invest. 103, 1287-1298 (1999); Bjorkegren, J., Beigneux, A., Bergo, M. O., Maher, J. J. & Young, S. G. J. Biol. Chem. 277, 5476-5483 (2002)). The Mx1 promoter is induced by interferon-inducing substances such as polyinosinic-polycytidylic acid (pIpC) (Raabe, M. et al. J. Clin. Invest. 103, 1287-1298 (1999); Bjorkegren, J., Beigneux, A., Bergo, M. O., Maher, J. J. & Young, S. G. J. Biol. Chem. 277, 5476-5483 (2002); Ralf, K., Schwenk, F., Aguet, M. & Rajewsky, K. Science. 269, 1427-1429 (1995)) such that treatment of MTPp^{flox/flox}Mx1Cre mice with pIpC leads to deletion of the MTPp gene (MTPp^{Δ/Δ}) primarily within the liver, spleen and intestine providing a degree of tissue specificity to the gene deletion. As previously reported, treatment of 6-10 week old MTPp^{flox/flox}Mx1Cre mice (or MTP^{flox/flox}Mx1Cre mice) with pIpC caused progressive hepatic steatosis (1C) and decreases in serum triglyceride and cholesterol levels (Table 5) in association with decreased MTP mRNA levels as defined by RT-PCR (FIG. 1D) and protein levels (FIG. 1E) in liver and colon.

Given the detection of a biochemical association between CD1d and MTP (FIG. 1A), it was hypothesized that MTP-deficiency induced by Mx-1 regulated Cre activity would affect CD1d expression and/or function in the hepatocyte in vivo. Therefore, the expression and distribution of CD1d in the liver of MTP^{flox/flox}Mx1Cre mice before and after pIpC treatment was examined. Prior to pIpC treatment, CD1d was present diffusely throughout the hepatocyte with a membranous staining pattern as shown by the green fluorescence (FIG. 1B, panel a). In comparison, after pIpC treatment, CD1d in hepatocytes from MTPp^{Δ/Δ} mice was noted to retreat into a prominent perinuclear staining pattern with reduced cell surface expression (FIG. 1B, panel b). In contrast, pIpC treatment of MTP^{flox/flox}Mx1Cre mice did not affect the distribution of either phalloidin staining of actin (FIG. 1B, panel b) or staining for carcinoembryonic antigen cell adhesion molecule 1 expression, a constitutive cell surface molecule on hepatocytes (FIG. 1B, panels c and d). These results indicate that the intracellular trafficking of CD1d is selectively influenced by the presence of MTP within hepatocytes in vivo.

TABLE 5
Characteristics of MTP^{flox/flox}Mx1Cre and WT C57BL/6J mice after pIpC
treatment.^{a}
1 day after treatment

| | MTP^{flox/flox}Mx1Cre (n = 9) | MTP^{flox/flox}Mx1Cre + pIpC (n = 9) | p value^{b} |
|---|---|---|---|
| Total S. Cholesterol^{c} | 144.0 ± 8.9 | 55.1 ± 9.7 | 0.02 |
| (mg/dl) | | | |
| Serum triglycerides | 62.6 ± 4.6 | 52.0 ± 7.5 | 0.25 |
| (mg/dl) | | | |
| Body weight (g) | 20.5 ± 0.6 | 21.8 ± 1.0 | 0.97 |
| | C57BL/6J (n = 10) | C57BL/6J + pIpC (n = 10) | p value |
| Total S. Cholesterol | 108.5 ± 12.0 | 96.2 ± 12.2 | 0.07 |
| Serum triglycerides | 112.1 ± 15.0 | 88.1 ± 6.6 | 0.91 |
| Body weight | 22.4 ± 0.6 | 21.5 ± 0.7 | 0.37 |

10 days after treatment

| | MTP^{flox/flox}Mx1Cre (n = 10) | MTP^{flox/flox}Mx1Cre + pIpC (n = 10) | p value |
|---|---|---|---|
| Total S. Cholesterol | 116.0 ± 8.6 | 49.2 ± 3.1 | 0.001 |
| Serum triglycerides | 63.2 ± 6.2 | 43.5 ± 7.3 | 0.02 |
| Body weight | 20.5 ± 0.6 | 21.8 ± 1.0 | 0.34 |
| | C57BL/6J (n = 8) | C57BL/6J + pIpC (n = 8) | p value |
| Total S. Cholesterol | 131.0 ± 9.5 | 127.0 ± 5.8 | 0.22 |
| Serum triglycerides | 131.5 ± 18.6 | 110.0 ± 11.2 | 0.94 |
| Body weight | 22.4 ± 0.6 | 20.7 ± 0.7 | 0.08 |

^{a}Female 6-8 week-old mice 1 day (A) or 10 days (B) after either vehicle or pIpC treatment were analyzed.
Data represent means ± SE.
^{b}P-values were calculated by two tailed unpaired t-test.
^{c}Total serum cholesterol.

### EXAMPLE 2

### Deletion of MTPp Gene Inhibits CD1d-Restricted Presentation by Hepatocytes

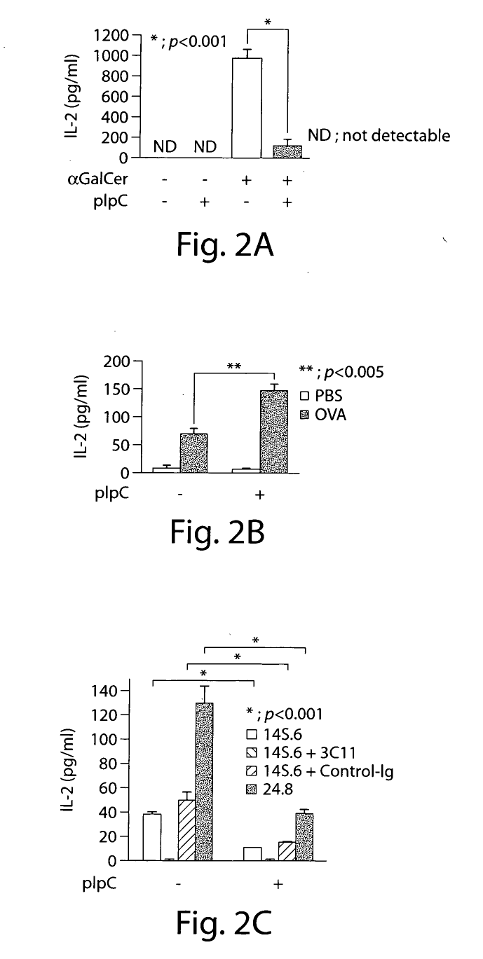

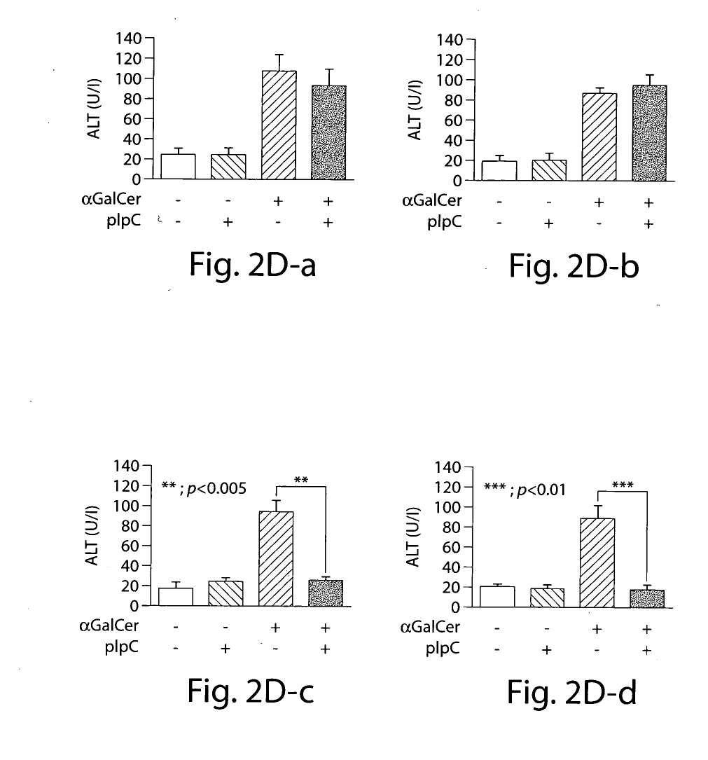

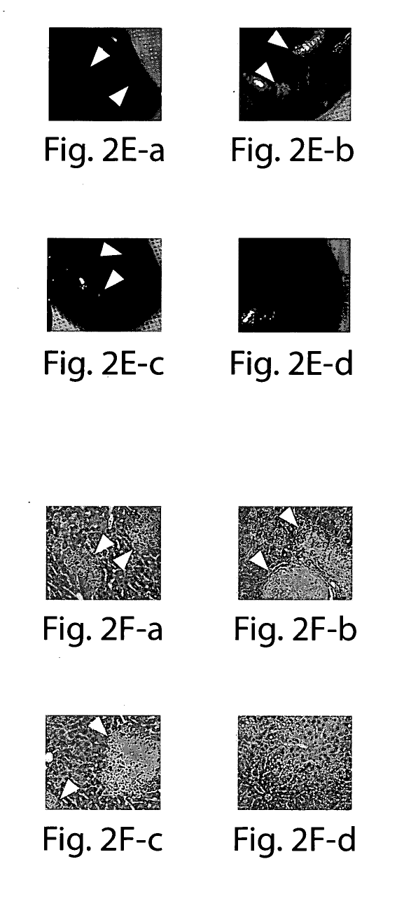

The effect of MTPp gene deletion upon the ability of CD1d expressed by hepatocytes to activate iNKT cells was determined. Hepatocytes obtained from either pIpC treated or untreated MTP^{flox/flox}Mx1Cre mice were therefore co-cultured with a mouse CD1d-restricted iNKT cell hybridoma, DN32.D3. As shown in FIG. 2A, hepatocytes from untreated MTP^{flox/flox}Mx1Cre mice stimulated the DN32.D3 cells to secrete interleukin-2 (IL-2) in the presence but not absence (vehicle control) of α-galactosylceramide (αGalCer). In contrast, hepatocytes from mice in which the MTPp gene had been deleted by pIpC treatment, stimulated little IL-2 production by the DN32.D3 cell line even in the presence of αGalCer (FIG. 2A), indicating a direct demonstration that MTP in hepatocytes is linked to the regulation of CD1d-restricted T cell responses by this cell type. Significantly, this effect was specific for CD1d since MHC class II-restricted activation of OVA-specific CD4⁺ T cells from OT-II transgenic mice by CD11c⁺ spleen cells from MTP^{flox/flox}Mx1Cre mice was unaffected by deletion of the MTPp gene.

### EXAMPLE 3

### Deletion of MTPp Gene Inhibits αGalCer-Induced Hepatitis

The clinical response to a hepatocyte mediated, CD1d-dependent response in vivo was evaluated (Osman, Y. et al. Eur. J. Immunol. 30, 1919-1928 (2000)). As would be expected, administration of αGalCer prior to pIpC treatment induced hepatitis in both wild-type (WT) C57BL/6J (B6) mice (FIG. 2B, panels a and b) and MTP^{flox/flox}Mx1Cre mice (FIG. 2B, panels c and d). The absence of inflammation with pIpC treatment alone is consistent with the lack of hepatic inflammation in either the absence per se of the MTPp gene (Raabe, M. et al. J. Clin. Invest. 103, 1287-1298 (1999)). or the presence of Mx1 activity induced by interferons (FIG. 2B, panels a-d) (Ralf, K., Schwenk, F., Aguet, M. & Rajewsky, K. Science. 269, 1427-1429 (1995)). In contrast to what would be predicted by the presence of hepatic steatosis induced by the lack of MTP function (Bjorkegren, J., Beigneux, A., Bergo, M. O., Maher, J. J. & Young, S. G. J. Biol. Chem. 277, 5476-5483 (2002)), MTPp^{Δ/Δ} mice were nearly completely protected from the inflammatory effects of αGalCer administration at either one day (FIG. 2D, panel c) or 10 days (FIG. 2B, panel d) after completion of the pIpC treatment regimen. The latter was evident from the absence of elevations of transaminases (FIG. 2D, panels c and d), macroscopic liver necrosis (FIG. 2E, panel d) or microscopic evidence of hepatitis (FIG. 2F, panel d) in the MTP^{flox/flox}Mx1Cre mice that received αGalCer with prior pIpC treatment. In contrast, WT B6 mice that received αGalCer at both one day (FIG. 2D, panel a) and 10 days (FIG. 2D, panel b) after pIpC treatment exhibited severe hepatitis. These results indicate in the absence of MTP, the liver is protected from CD1d-restricted and iNKT cell-mediated hepatocyte injury.

Significantly, this effect was specific for CD1d since MHC class II-restricted activation of OVA-specific CD4⁺ T cells from OT-II transgenic mice by CD11c⁺ dendritic cells obtained from the liver of MTP^{flox/flox}Mx1Cre mice was unaffected by deletion of the Mttp gene and, in fact, was increased consistent with the effects of interferon-α induced by pIpC treatment (FIG. 2D). CD1d-restricted autoreactivity to hepatocytes was also defective in the absence of MTP and affected T cells bearing either an invariant (24.8) or noninvariant (14S.6) TCR-α chain (FIG. 2C).

### EXAMPLE 4

### Inhibition of the MTPp Gene Inhibits CD1d-Restricted Antigen Presentation

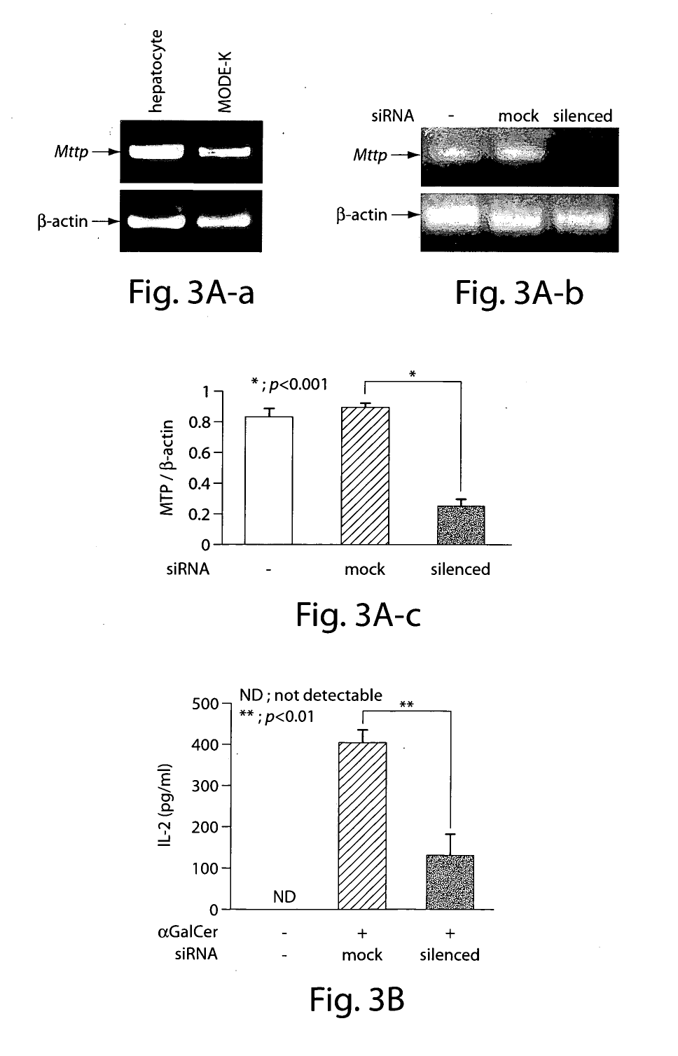

The relationship between MTP and CD1d in large intestinal epithelial cells (IECs) was determined. As predicted (Gordon, D. A., Wetterau, J. R. & Gregg, R. E. Trends Cell. Biol. 5, 317-321 (1995)), MTP was expressed by a mouse IEC line, MODE-K (FIG. 3A, panel a), which is also known to functionally express CD1d (van de Waal, Y. et al. Gastroenterology. 124, 1420-1431, (2003)). Gene silencing of MTPp expression in MODE-K cells with siRNA oligomers specific for MTPp transcripts caused a significant reduction in MTPp mRNA levels in the MODE-K cell line (FIG. 3B, panels b and c). In the absence of an exogenous source of the glycolipid antigen αGalCer, the MODE-K cell line was incapable of stimulating IL-2 production by DN32.D3 cells (FIG. 3B). In contrast, in the presence of αGalCer, the MODE-K cell line stimulated significant IL-2 production by the DN32.D3 cell line (FIG. 3B). This production of IL-2 was inhibited by silencing MTPp gene products using siRNA-specific oligomers but not siRNA oligomers directed at an irrelevant gene target (FIG. 3B). These studies indicate that MTP regulates the ability of IECs to exhibit CD1d-restricted antigen presentation.

### EXAMPLE 6

### Inhibition of Oxazolone Induced Colitis in Microsomal Triglyceride Transferase Deficient Mice

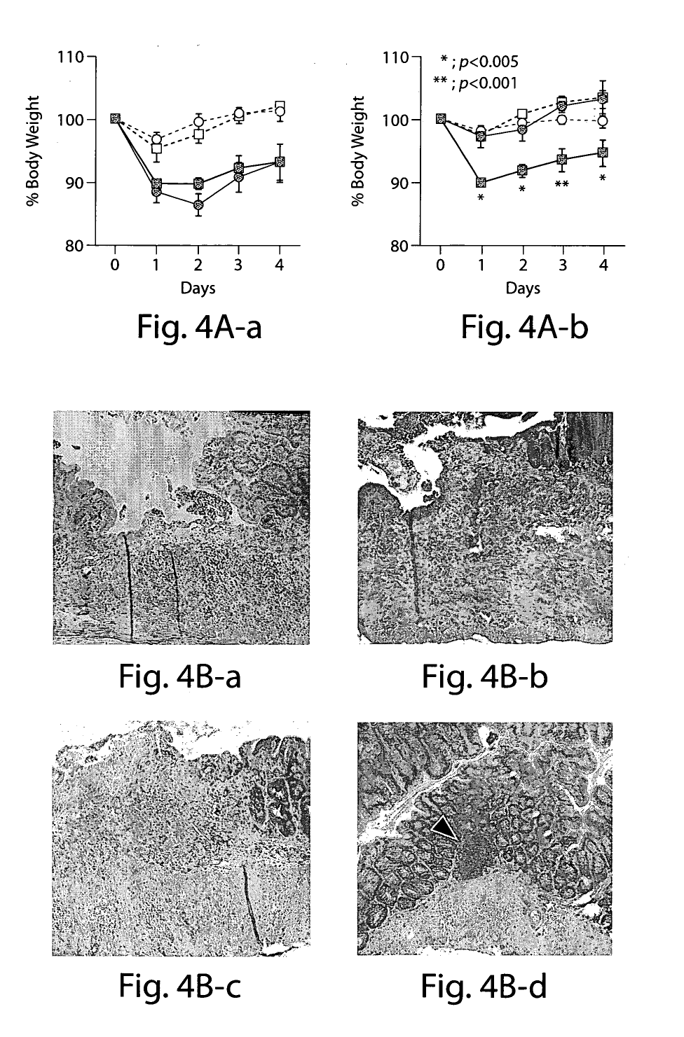

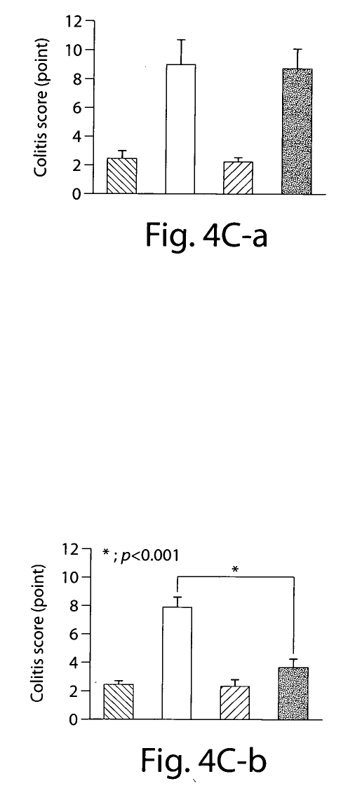

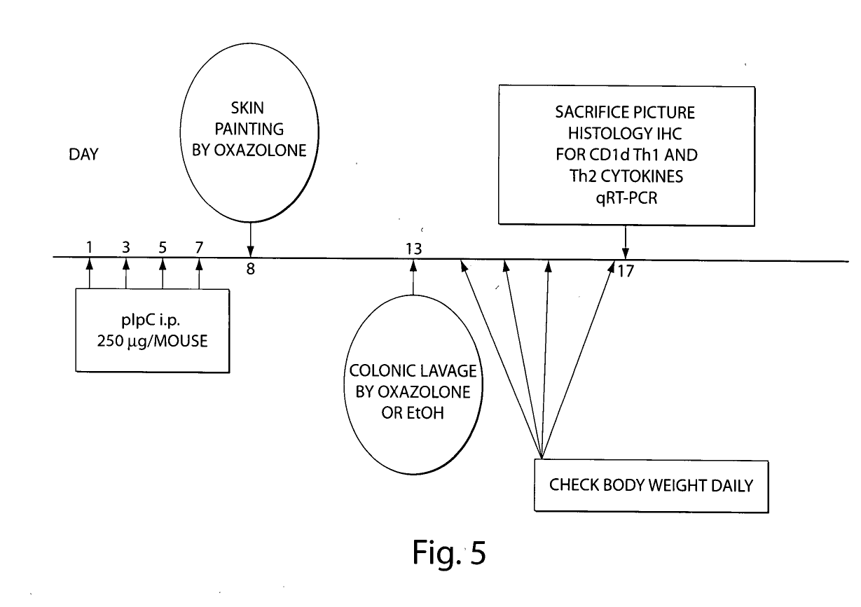

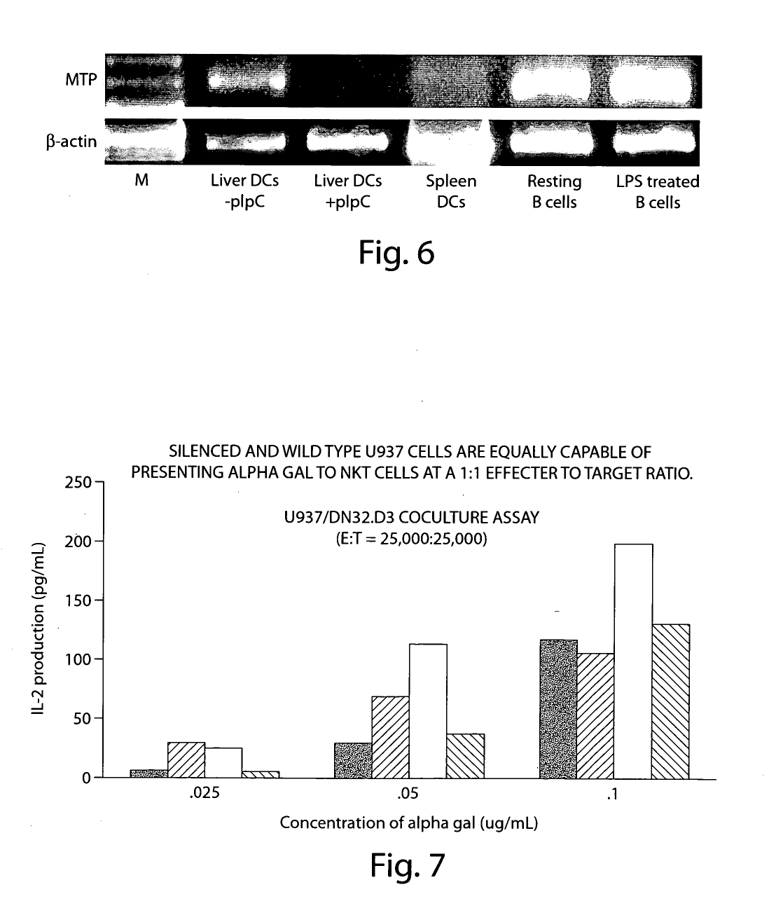

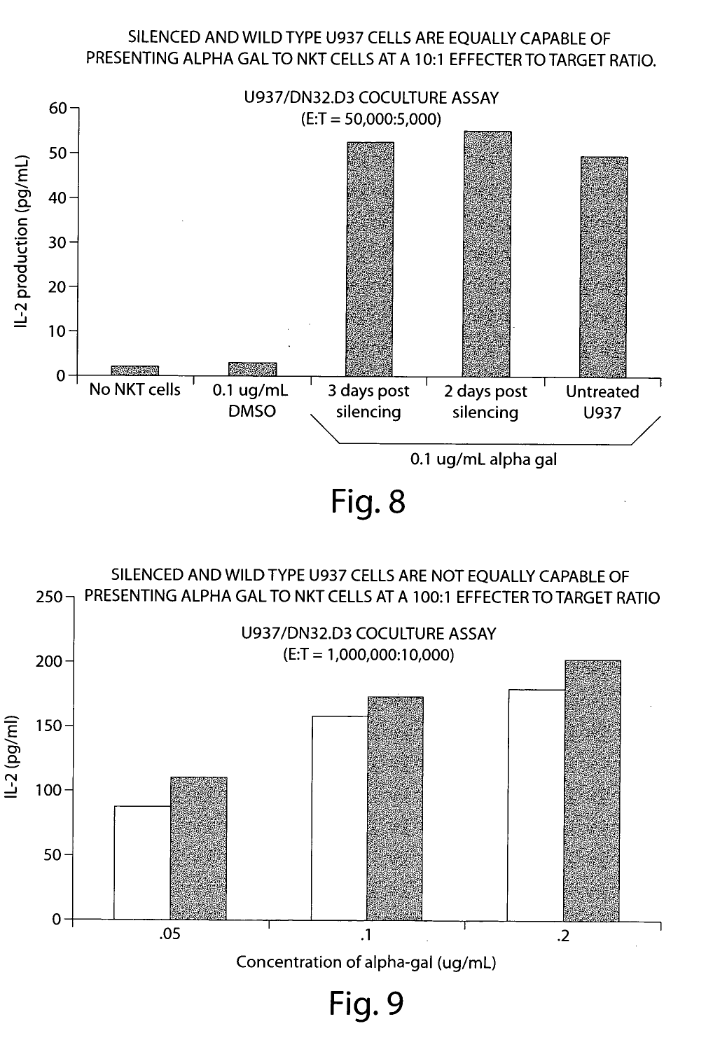

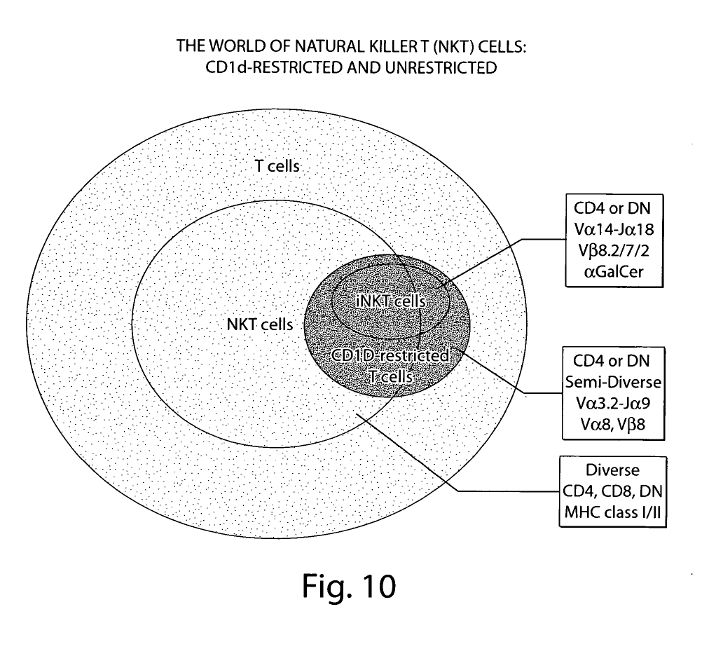

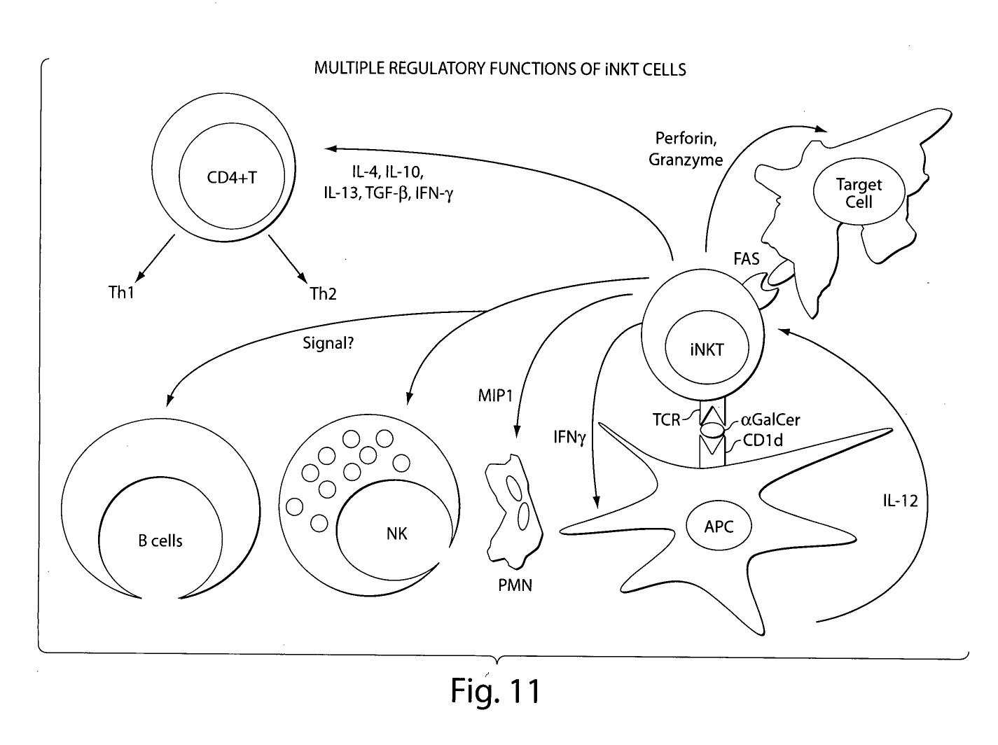

The effect of MTPp gene deletion on the clinicopathologic outcome of oxazolone-induced colitis was examined. This model of colitis has recently been shown to be mediated by CD1d and CD1d-restricted iNKT cells (Heller, F., Fuss, I. J., Nieuwenhuis, E., Blumberg, R. S. & Strober, W. Immunity. 17, 629-638 (2002)). Whereas MTP^{flox/flox}Mx1Cre mice experienced severe colitis in association after administration of the hapten oxazolone, as manifest by profound weight loss (FIG. 4A, panel b, closed squares) and pathological evidence of mucosal ulcerations and infiltration of intestinal tissues by inflammatory cells (FIG. 4B, panel c), MTPp^{Δ/Δ} mice in which the MTPp gene had been deleted by administration of pIpC exhibited minimal weight loss, which was identical to that of the MTP^{flox/flox}Mx1Cre mice exposed to the ethanol control (FIG. 4A, panel b, closed circles) and little histologic evidence of colitis (FIG. 4B, panel d). A semiquantitative estimate of colitis severity confirmed these results (FIG. 4C, panel b). Specifically, MTP^{flox/flox}Mx1Cre mice exhibited little evidence of colitis in response to the administration of oxazolone when pretreated with pIpC. Given that colitis was not ameliorated in WT B6 mice exposed to oxazolone in the presence of pIpC administration at the same schedule administered to the MTP^{flox/flox}Mx1Cre mice (FIG. 4A-C), these results indicate that the protection observed in the MTPp^{Δ/Δ} mice was not simply due to the effects of the pIpC treatment.

### EXAMPLE 7

### MTP is Present and Functional in Antigen Presenting Cells

Since MTP plays a role in CD1d antigen presentation by hepatocytes and IECs, it was hypothesized that MTP may serve as a chaperone for CD1d in all CD1d-positive cell types. MTP expression had never been detected in professional APCs, and most research in the field has focused on the functions of MTP in the liver and intestine. Mttp transcript expression was examined in a variety of potential APC types (FIG. 15A). Mttp was detected in mouse primary tissues (liver, heart, lung, ovaries, peritoneal exudate cells, small intestine, colon, spleen, thymus, lymph node, B cells, liver dendritic cells, bone marrow dendritic cells), mouse thymus (RMAS), APC (RAW) and NKT cell (DN32) lines, and in human B, T, and monocyte cell lines (C1Rd, Jurkat, U937). Mttp was not present in DCs isolated from MTP gene deleted mice, but was present at low abundance in every other immunologic and nonimmunologic tissue examined. Mttp expression in intestine was notably increased relative to APCs as would be expected from a tissue actively secreting lipoproteins.

To verify the presence of MTP protein in APCs, we performed subcellular fractionation on primary mouse splenocytes. Wild type mice were injected with a flt3L secreting melanoma to increase the number of splenic dendritic cells as has been previously described. Splenocytes were subjected to subcellular fractionation to obtain protein from mitochondria and microsomes. As shown in FIG. 15B, a 97-kDa band consistent with MTP could be detected in the microsomal but not mitochondrial fraction by immunoblotting with an MTP-specific antibody.

To demonstrate that MTP is functional in APCs, a previously described triglyceride transfer assay was used. In this assay, MTP function is assessed by studying the transfer of fluorescently labeled triglycerides embedded in donor phospholipid vesicles to acceptor vesicles. During the transfer, the amount of triglyceride associated with MTP can be measured as an increase in fluorescence over time. Splenocyte lysates contained measurable triglyceride transfer activity, which could be abrogated by addition of BMS 197636 (9-[4-[[[4′-(trifluoromethyl)[1,1′-biphenyl]-2-yl]carbonyl]amino]butyl-piperidin-4-yl]-N-(2,2,2-trifluoroethyl)-9H-fluorene-9-carboxamide), a specific chemical inhibitor of MTP lipid transfer (FIG. 15C). Specific activity in the splenocyte lysates was 0.04% triglyceride being transferred per μg of lysate protein per hour. In addition, the CD1d-positive mouse NKT cell line DN32 that was shown to express mttp transcripts (FIG. 15A) exhibited a specific activity of 0.22%. In comparison, rat liver microsomes exhibited a specific activity of 4.78%.

### EXAMPLE 8

### MTP Can Transfer a Lipid to Recombinant CD1d In Vitro

MTP could directly transfer lipids to CD1d, or it could indirectly influence CD1d presentation such as through a lipoprotein intermediate. To test for direct lipid transfer from MTP to CD1d, a reductionist in vitro approach was employed using phosphatidyl ethanolamine (PE) as a model lipid antigen. PE has been shown to bind to CD1d and can be recognized by several invariant NKT cell hybridomas. PE is also a known substrate for MTP.

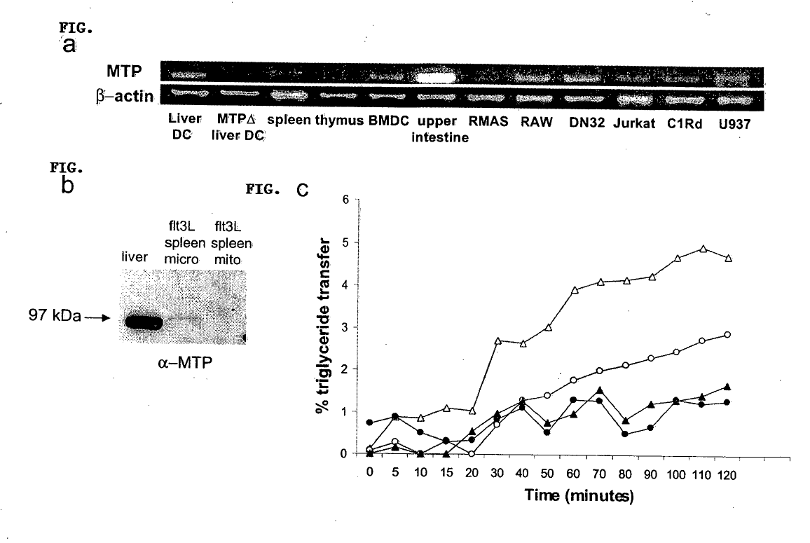

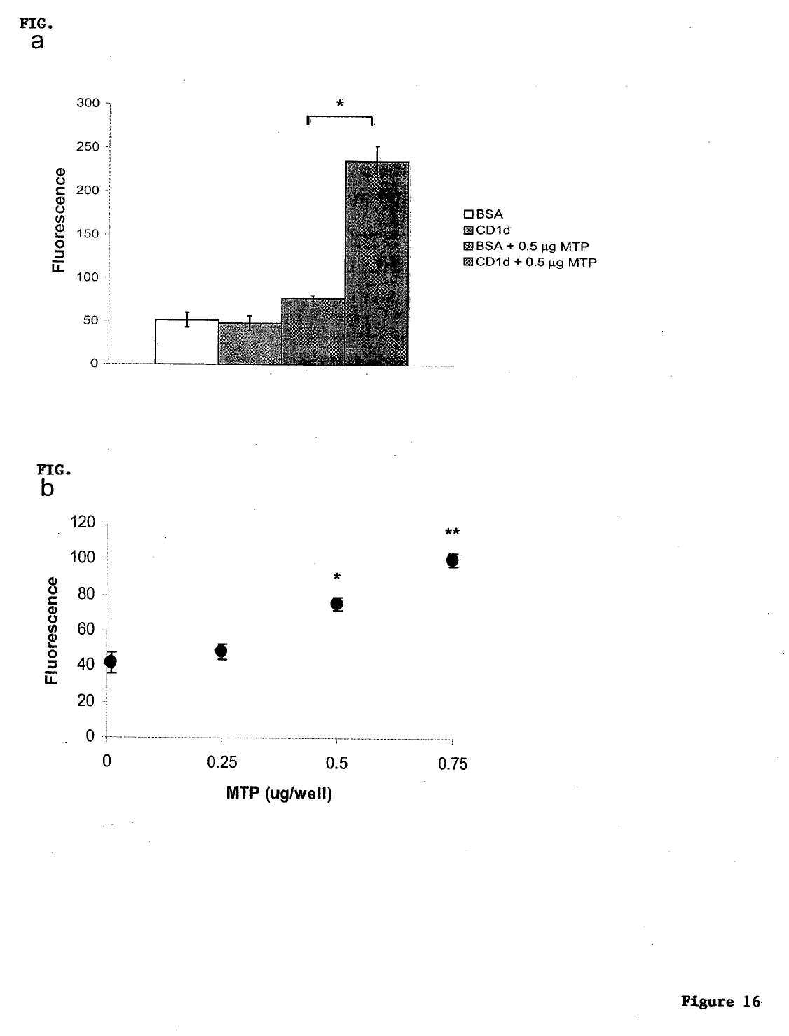

Recombinant β2m associated CD1d was coated onto a 96 well plate and incubated with liposomes containing a 6:1 ratio of unlabeled PE:NBD-labeled PE. The fluorescent NBD label was conjugated to the PE headgroup in order to avoid steric interference between PE acyl tails and CD1d hydrophobic pockets. MTP purified from rat liver homogenates was added to the CD1d or BSA coated wells at a 1:10 MTP:CD1d molar ratio, incubated at 37 degrees for 2 hours and then washed with PBS. Lipids bound to CD1d were eluted with isopropanol, transferred to a black microtiter plate, and the level of fluorescence determined. As can be seen in FIG. 16A, in the presence of MTP, PE was transferred to CD1d, but not to BSA, as seen by a 5-fold increase in fluorescence above background. In FIG. 16B, the amount of fluorescent PE transferred to CD1d increased with increasing concentrations of purified MTP. Assuming equal efficiency of MTP-mediated transfer of labeled versus unlabeled PE to the CD1d pocket and that all CD1d molecules bound to the plastic in an orientation with the groove solvent accessible, we calculated that the maximum limit of NBD-labeled PE that could be transferred to CD1d was 4.7 picomoles in this assay. Given that the observed values of MTP-mediated PE transfer to CD1d varied from 0.4 to 1.6 picomoles of fluorescence in all replications associated with FIG. 16A, it was estimated that one-tenth to one-third of the CD1d molecules were able to accept PE in the presence of 0.5 μg MTP.

### EXAMPLE 9

### In Vitro Inhibition of MTP Reduces CD1d-Mediated Antigen Presentation of Dendritic Cells

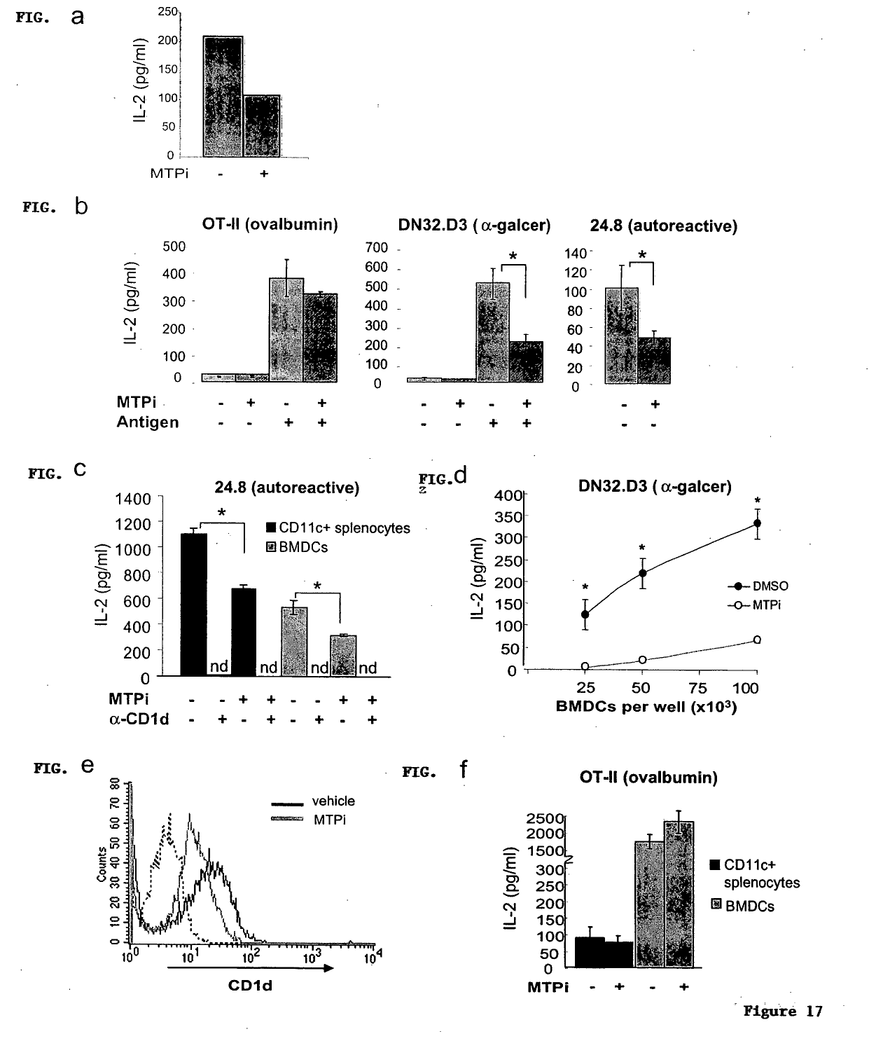

9-[4-[2,5-Dimethyl-4-[[[4′-(trifluoromethyl)[1,1′-biphenyl]-2-yl]carbonyl]amino]-1H-benzimidazol-1-yl]butyl]-N-(2,2,2-trifluoroethyl)-9H-fluorene-9-carboxamide (referred to herein as BMS212122) is a specific chemical inhibitor of MTP that blocks transfer of triglycerides, phospholipids, and cholesterol esters in vitro and prevents secretion of apoB from HepG2 cells. CD1d presentation in the mouse epithelial cell line, MODE-K, is regulated by MTP. As shown in FIG. 17A, incubation of MODE-K cells with BMS212122 decreased the presentation of α-galcer to DN32 cells. These studies indicate that the MTP inhibitor BMS212122 blocks CD1d function similar to its previously established role in inhibiting apoB secretion.

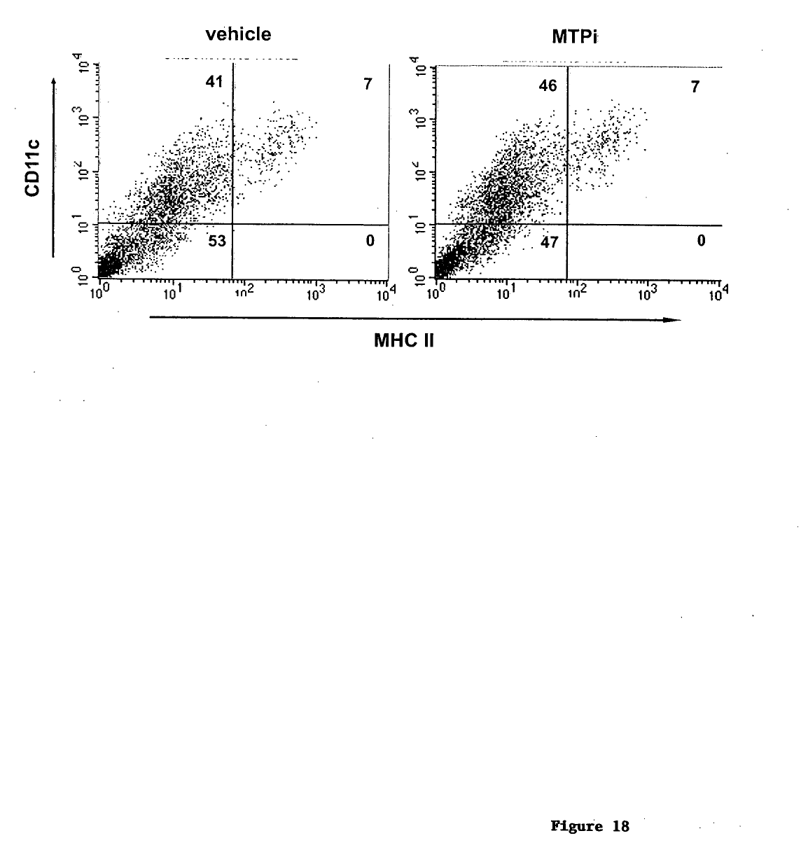

The role of MTP in CD1d presentation by primary mouse APCs was examined. Splenocytes were isolated from wild type mice and assayed for their ability to present an exogenous antigen, α-galcer, to DN32 NKT cells and their ability to stimulate the autoreactive NKT cell line 24.8. When splenocytes were incubated with BMS212122 for 24 hours prior to addition of NKT cells, their ability to present exogenous and endogenous CD1d-restricted antigens was reduced (FIG. 17B). BMS212122 exhibited no effect, however, on splenocyte presentation of ovalbumin to the MHC class II-restricted T cell line, OT-II (FIG. 17B). Dendritic cells were then isolated from wild type splenocytes by positive selection on CD11c magnetic beads. The effect of MTP inhibition on presentation of an endogenous ligand to the 24.8 NKT cells was highly pronounced in the CD11c-positive dendritic cell population (FIG. 17C). Dendritic cells from mouse bone marrow (BMDC) were cultured with BMS212122 or vehicle control after 6 days of differentiation in the presence of GM-CSF. On day 10, BMDCs were collected and cocultured with NKT cells. BMS212122 had a significant effect on both presentation of an endogenous ligand to autoreactive 24.8 cells (FIG. 17C) and on presentation of α-galcer to DN32 cells (FIG. 17D). MHC class II and CD11c surface staining were unaffected by BMS212122 indicating no deleterious effects on maturation of BMDCs (FIG. 18). In contrast, a decrease in CD1d surface expression on BMDCs was observed over time in the presence of BMS212122. One day after addition of BMS212122, CD1d expression on BMDCs was 86% of the level observed in vehicle treated controls, and after 4 days of BMS212122 treatment CD1d expression was decreased to 66% of the vehicle controls. FIG. 17E shows CD1d surface expression 4 days after BMS212122 addition.

The MHC II pathway was intact in BMS212122-treated cells; CD11c-positive splenic DCs and BMDCs exhibited efficient presentation of ovalbumin to CD4⁺, MHC class II-restricted ovalbumin-specific T cells (FIG. 17F). Thus, MTP inhibition resulted in a selective defect in CD1d-mediated antigen presentation.

### EXAMPLE 10

### Dendritic Cells Isolated from MTP Gene Deleted Mice are Defective in Presenting CD1d-Restricted Antigens Ex Vivo

Since deletion of MTP is embryonic lethal, mice have been previously generated in which the mttp gene has been flanked by loxp sites. These Mttp ‘floxed’ mice were crossed to mice transgenic for cre recombinase under the control of the interferon responsive Mx1 promoter. The resulting MTPmx1 mice delete mttp after a series of injections with the TLR3 ligand pIpC⁴⁴. Deletion has been shown to be efficient in the liver and spleen, and a severe reduction in mttp transcript levels in liver and splenic DC was confirmed by RT-PCR analysis (FIG. 15A).

Flow cytometric analysis of splenocytes isolated from MTP gene deleted mice displayed an approximately 30% reduction in the surface expression of CD1d in the absence of MTP (Table 6). No reduction in CD1d expression was observed in control wild-type mice injected with pIpC arguing against nonspecific effects of pIpC treatment.

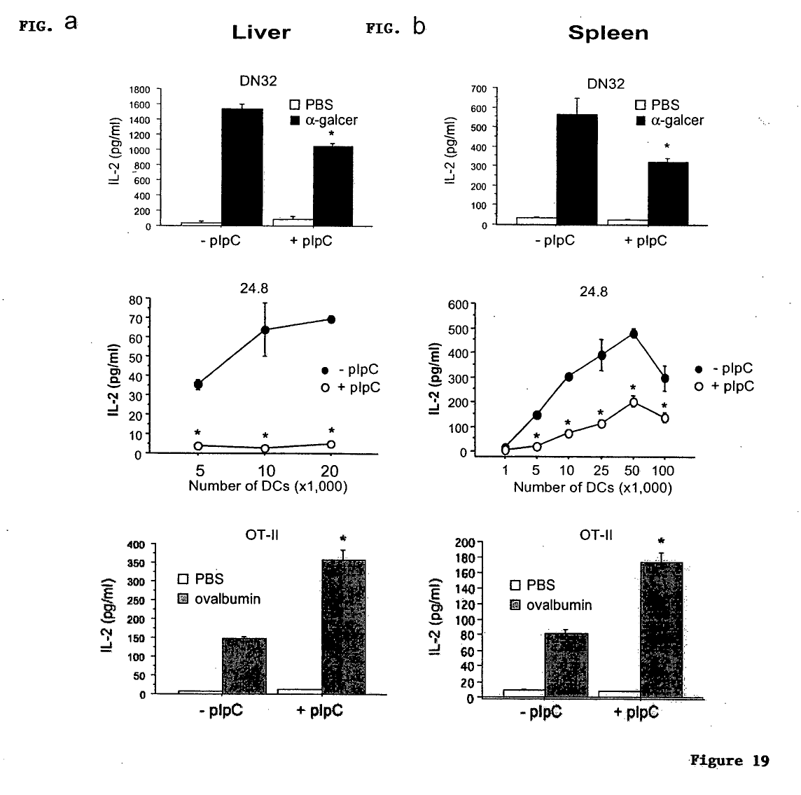

CD11c⁺ DCs were isolated from the liver and spleen of MTPmx1 mice that had been treated or not treated with pIpC to induce MTP gene deletion. Liver (FIG. 19A, top) and spleen (FIG. 19B, top) DCs from the gene deleted mice exhibited a significant reduction in their ability to present α-galcer to DN32 cells. Likewise, the MTP gene deleted liver and spleen DCs failed to stimulate the autoreactive NKT cell line 24.8 (middle panels, FIGS. 19A and 19B). The presentation of ovalbumin on MHC class II was increased on liver and spleen dendritic cells from pIpC treated animals as expected given the ability of TLR3 ligands to activate DCs (bottom panels, FIGS. 19A and 19B).

TABLE 6

| Genotype | % CD1d+ | Mean fluorescence intensity | n |
|---|---|---|---|
| MTPmx1 + pIpC | 29.9 +/− 4.7* | 6.2 +/− 0.9* | 6 |
| MTPmx1 − pIpC | 42.1 +/− 4.5 | 8.4 +/− 0.8 | 5 |
| B6 + pIpC | 42.2 | 8.0 | 1 |
| B6 − pIpC | 43.3 | 8.2 | 1 |

*p < 0.005 compared to MTPmx1 − pIpC
n, number of mice examined

### EXAMPLE 11

### MTP is Critical for CD1d-Restricted Antigen Presentation in Human Cells

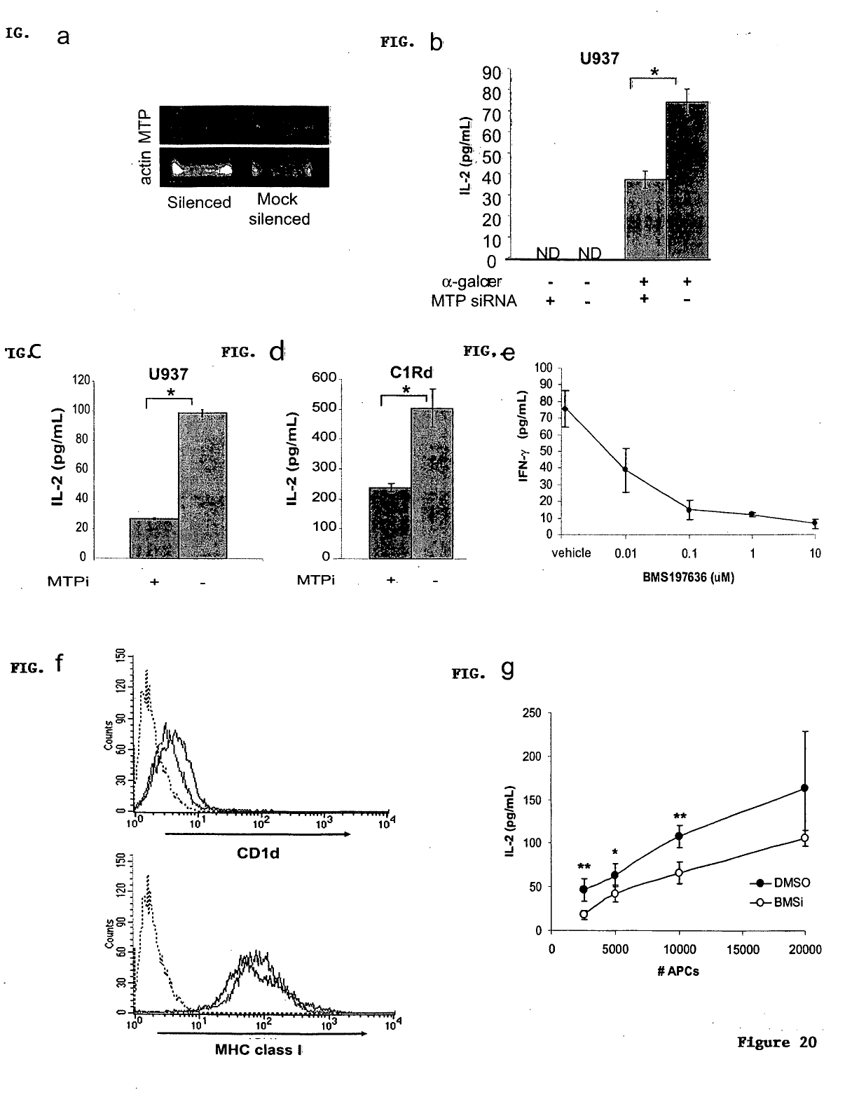

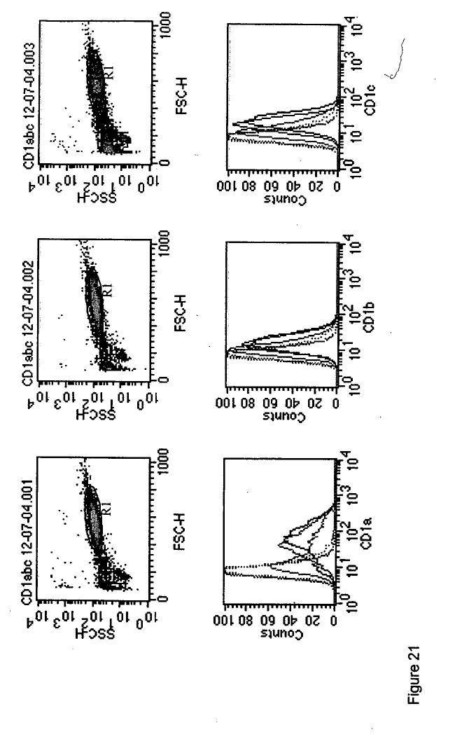

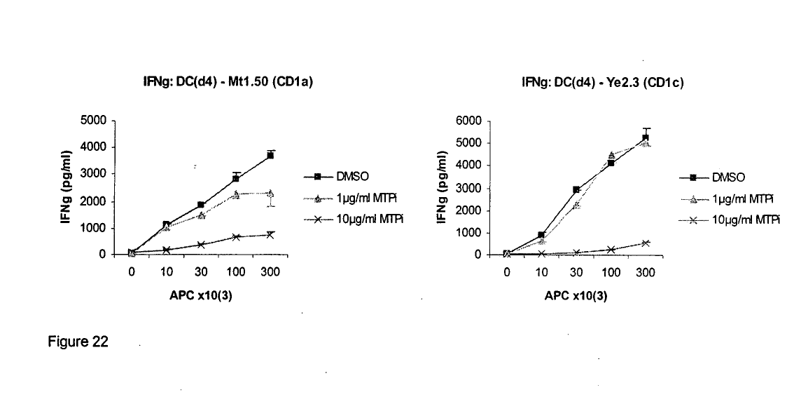

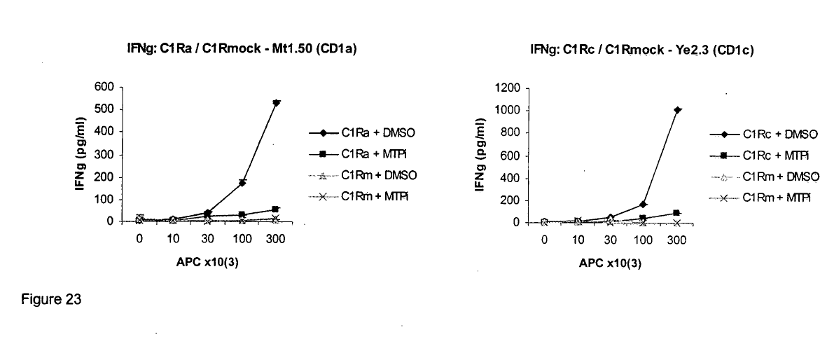

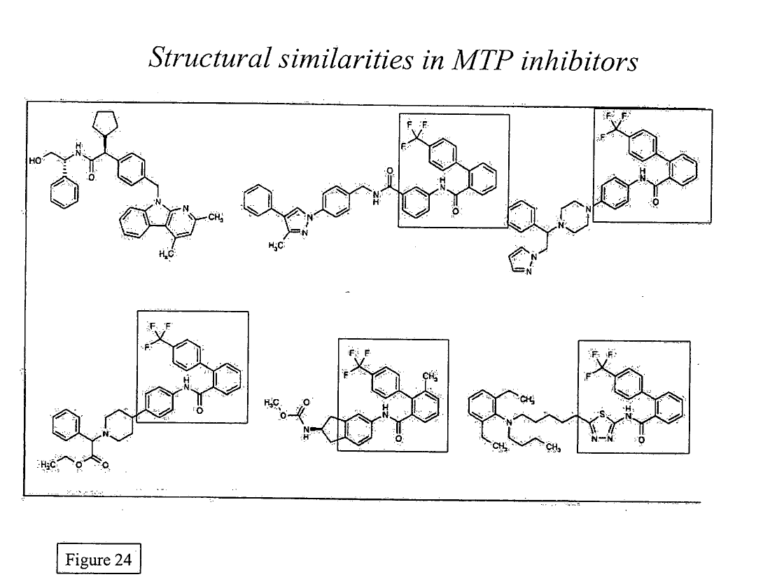

MTP is highly conserved among mammalian species with an 86% identity between mouse and human MTP at the amino acid level. It was therefore hypothesized that MTP inhibition in human antigen presenting cells would affect CD1d antigen presentation. To test this, MTP expression was silenced in the human monocyte cell line U937. U397 cells express MTP (FIG. 15A) and low levels of surface CD1d but are highly potent APCs for CD1d-restricted NKT cells. U937 cells that were transfected with human mttp specific siRNA oligomers displayed a 48% reduction in mttp transcript levels compared to cells transfected with irrelevant oligomers (mock) after 48 hours as shown in FIG. 20A. The silenced and mock silenced U937 cells were incubated with α-galcer for 3 hours, washed, and cocultured with the iNKT cell line DN32. NKT cell activation was measured by IL-2 production, and, as shown in FIG. 20B, the mttp-silenced U937 cells exhibited a significant reduction in their ability to present the model CD1d-restricted exogenous antigen. A similar reduction in IL-2 release from DN32 cells was observed when U937 cells were cultured with α-galcer and BMS212122 as compared to the vehicle control (FIG. 20C).

C1Rd is a human B cell line transfected with human CD1d. C1Rd cells were cultured with MTP inhibitor or vehicle for 5 days, pulsed with α-galcer, fixed with glutaraldehyde and washed prior to coculture with DN32 NKT cells. Incubation of C1Rd cells with BMS212122, but not the vehicle, inhibited the presentation of α-galcer as determined by a reduction in IL-2 secretion (FIG. 20D). To exclude nonspecific effects of BMS212122, C1Rd cells were incubated with a second MTP chemical inhibitor, BMS197636, which resulted in dose-dependent inhibition of CD1d-mediated activation of a human peripheral blood derived iNKT cell line in the presence of a limiting concentration of PMA (FIG. 20E). Thus MTP inhibition in C1Rd cells resulted in a diminished ability to present CD1d-restricted antigens to mouse and human iNKT cells.

To further determine the effect of MTP inhibition in primary human antigen presenting cells, dendritic cells (moDC) were differentiated from CD14-positive peripheral blood mononuclear cells obtained from healthy volunteers. Briefly, monocytes were selected on CD14 magnetic beads and cultured in media supplemented with autologous plasma, GM-CSF, IL-4, and either BMS212122 or vehicle. After 5 days in the presence of BMS212122, the moDCs had upregulated MHC class I to an equal extent in comparison to vehicle treatment (mean fluorescence intensity 76 vs. 70; BMS212122 vs. vehicle); however, moDCs cultured in the presence of BMS212122 expressed significantly lower levels of surface CD1d (mean fluorescence intensity 3.3 vs. 4.2; p<0.05) (FIG. 20F). As can be seen in FIG. 20G the MTP inhibited moDCs also displayed a reduced capacity to present α-galcer to DN32 iNKT cells.

Other embodiments are within the following claims.

1. A method of reducing lipidation of a CD1 molecule in a cell, comprising contacting said cell with a microsomal triglyceride transfer protein (MTP) inhibitor.

2. The method of claim 1, wherein said CD1 is CD1a, CD1b, CD1c, CD1d or CD1e.

3. The method of claim 1, wherein said MTP inhibitor reduces the direct transfer of a phospholipid or a glycolipid from MTP to CD1.

4. The method of claim 1, wherein said MTP inhibitor preferentially reduces transfer of a CD1-binding lipid compared to a triglyceride.

5. The method of claim 4, wherein said CD1-binding lipid is a phospholipid, a glycolipid, a lipoarabino mannin, a monoglucosides or a lipoprotein.

6. The method of claim 1, wherein said cell is a CD1-expressing cell.

7. The method of claim 6, wherein said CD-1 expressing cell is a B cell, a monocyte, a macrophage, a dendritic cell, a hepatocyte, an epithelial cell, a thymocyte, or a T-cell.

8. The method of claim 1, wherein said MTP inhibitor is a compound according to Formula I:
I. 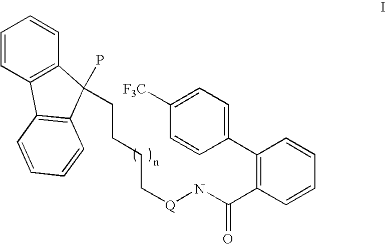
wherein n is zero or 1;
P is 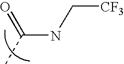
or a 5- or 6-membered heterocycle selected from the group consisting of: 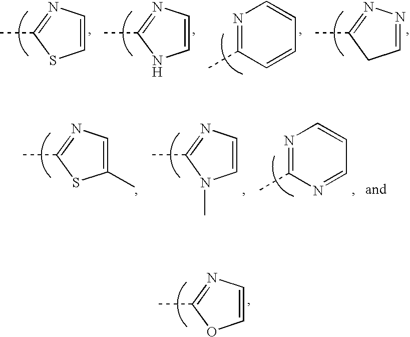
and Q is 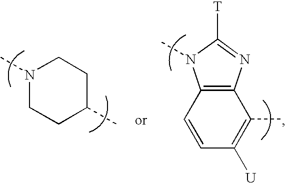
wherein T and U are, independently, hydrogen or lower alkyl.

9. The method of claim 1, wherein said MTP inhibitor is a compound according to Formula I:
I. 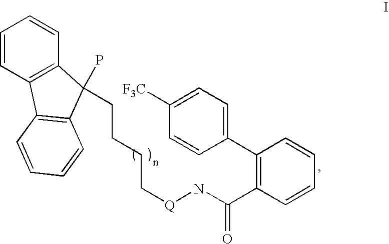
wherein n is zero or 1;
P is 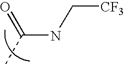
or a 5- or 6-membered heterocycle selected from the group consisting of: 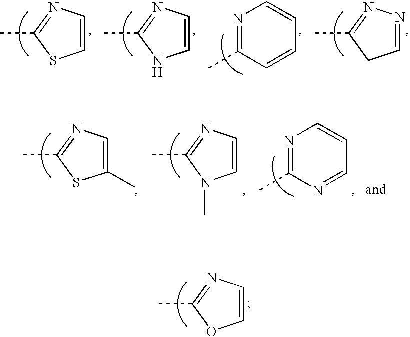
and Q is 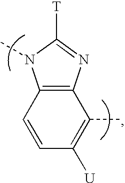
wherein T and U are, independently, hydrogen or lower alkyl.

10. The method of claim 9, wherein P is

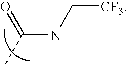

11. A method of claim 1, wherein said inhibitor preferentially associates with a phospholipid binding domain of MTP compared to a triglyceride binding domain of said MTP.

12. A method of inhibiting CD 1-mediated inflammation or immune function, comprising contacting a CD 1-expressing cell with an inhibitor of microsomal triglyceride transfer protein (MTP), wherein direct transfer of lipid to CD1 is reduced following contact of said CD1-expressing cell with the inhibitor of MTP.

13. The method of claim 12, wherein said CD1 expressing cell is a B cell, a monocyte, a macrophage, a dendritic cell, a hepatocyte, an epithelial cell, a thymocyte, or a T-cell.

14. The method of claim 12, wherein said lipid is a CD1-binding lipid.

15. The method of claim 14, wherein said CD 1-binding lipid is a phospholipid, a glycolipid, a lipoarabino mannin, a monoglucosides or a lipoprotein. a phospholipid

16. A method of inhibiting tissue inflammation comprising contacting a cell with a MTP inhibitor in an amount that inhibits MTP mediated transfer of a phospholipid to CD1.

17. A composition comprising a compound that preferentially associates with a phospholipid binding domain of a MTP compared to a triglyceride binding domain.

18. A method of inhibiting an association of MTP and CD1, comprising contacting a cell expressing said MTP and said CD1 with an MTP-binding compound, wherein said association is reduced in the presence of said compound compared to that in the absence of said compound.

19. A method of inhibiting an association of MTP and CD1, comprising contacting a cell expressing said MTP and said CD1d with an CD1-binding compound, wherein said association is reduced in the presence of said compound compared to that in the absence of said compound.

20. A method of increasing anti-tumor immunosurveillance, comprising identifying a subject suffering from or at risk of developing a tumor and administering to said subject a microsomal triglyceride transfer protein (MTP) inhibitor.

21. A method of increasing anti-tumor immunosurveillance comprising contacting a cell with a MTP inhibitor in an amount that inhibits the production of IL-13.


SEQUENCE LISTING (22 sequences)

SEQ ID NO: 1 - RNA, 21 nt, Artificial Sequence
  Description of Artificial Sequence chemically synthesized siRNA
aagcucuggaacuaccaacga

SEQ ID NO: 2 - RNA, 21 nt, Artificial Sequence
  Description of Artificial Sequence chemically synthesized siRNA
ucguugguaguuccagagcuu

SEQ ID NO: 3 - DNA, 3392 bp, Homo sapiens
actccctcactggctgccattgaaagagtccacttctcagtgactcctagctgggcactggatgcagttgaggattgctggtcaatatgattcttcttgctgtgctttttctctgcttcatttcctcatattcagcttctgttaaaggtcacacaactggtctctcattaaataatgaccggctgtacaagctcacgtactccactgaagttcttcttgatcggggcaaaggaaaactgcaagacagcgtgggctaccgcatttcctccaacgtggatgtggccttactatggaggaatcctgatggtgatgatgaccagttgatccaaataacgatgaaggatgtaaatgttgaaaatgtgaatcagcagagaggagagaagagcatcttcaaaggaaaaagcccatctaaaataatgggaaaggaaaacttggaagctctgcaaagacctacgctccttcatctaatccatggaaaggtcaaagagttctactcatatcaaaatgaggcagtggccatagaaaatatcaagagaggtctggctagcctatttcagacacagttaagctctggaaccaccaatgaggtagatatctctggaaattgtaaagtgacctaccaggctcatcaagacaaagtgatcaaaattaaggccttggattcatgcaaaatagcgaggtctggatttacgaccccaaatcaggtcttgggtgtcagttcaaaagctacatctgtcaccacctataagatagaagacagctttgttatagctgtgcttgctgaagaaacacacaattttggactgaatttcctacaaaccattaaggggaaaatagtatcgaagcagaaattagagctgaagacaaccgaagcaggcccaagattgatgtctggaaagcaggctgcagccataatcaaagcagttgattcaaagtacacggccattcccattgtggggcaggtcttccagagccactgtaaaggatgtccttctctctcggagctctggcggtccaccaggaaatacctgcagcctgacaacctttccaaggctgaggctgtcagaaacttcctggccttcattcagcacctcaggactgcgaagaaagaagagatccttcaaatactaaagatggaaaataaggaagtattacctcagctggtggatgctgtcacctctgctcagacctcagactcattagaagccattttggactttttggatttcaaaagtgacagcagcattatcctccaggagaggtttctctatgcctgtggatttgcttctcatcccaatgaagaactcctgagagccctcattagtaagttcaaaggttctattggtagcagtgacatcagagaaactgttatgatcatcactgggacacttgtcagaaagttgtgtcagaatgaaggctgcaaactcaaagcagtagtggaagctaagaagttaatcctgggaggacttgaaaaagcagagaaaaaagaggacaccaggatgtatctgctggctttgaagaatgccctgcttccagaaggcatcccaagtcttctgaagtatgcagaagcaggagaagggcccatcagccacctggctaccactgctctccagagatatgatctccctttcataactgatgaggtgaagaagaccttaaacagaatataccaccaaaaccgtaaagttcatgaaaagactgtgcgcactgctgcagctgctatcattttaaataacaatccatcctacatggacgtcaagaacatcctgctgtctattggggagcttccccaagaaatgaataaatacatgctcgccattgttcaagacatcctacgtttggaaatgcctgcaagcaaaattgtccgtcgagttctgaaggaaatggtcgctcacaattatgaccgtttctccaggagtggatcttcttctgcctacactggctacatagaacgtagtccccgttcggcatctacttacagcctagacattctctactcgggttctggcattctaaggagaagtaacctgaacatctttcagtacattgggaaggctggtcttcacggtagccaggtggttattgaagcccaaggactggaagccttaatcgcagccacccctgacgagggggaggagaaccttgactcctatgctggtatgtcagccatcctctttgatgttcagctcagacctgtcacctttttcaacggatacagtgatttgatgtccaaaatgctgtcagcatctggcgaccctatcagtgtggtgaaaggacttattctgctaatagatcattctcaggaacttcagttacaatctggactaaaagccaatatagaggtccagggtggtctagctattgatatttcaggtgcaatggagtttagcttgtggtatcgtgagtctaaaacccgagtgaaaaatagggtgactgtggtaataaccactgacatcacagtggactcctcttttgtgaaagctggcctggaaaccagtacagaaacagaagcaggcttggagtttatctccacagtgcagttttctcagtacccattcttagtttgcatgcagatggacaaggatgaagctccattcaggcaatttgagaaaaagtacgaaaggctgtccacaggcagaggttatgtctctcagaaaagaaaagaaagcgtattagcaggatgtgaattcccgctccatcaagagaactcagagatgtgcaaagtggtgtttgcccctcagccggatagtacttccagcggatggttttgaaactgacctgtgatattttacttgaatttgtctccccgaaagggacacaatgtggcatgactaagtacttgctctctgagagcacagcgtttacatatttacctgtatttaagatttttgtaaaaagctacaaaaaactgcagtttgatcaaatttgggtatatgcagtatgctacccacagcgtcattttgaatcatcatgtgacgctttcaacaacgttcttagtttacttatacctctctcaaatctcatttggtacagtcagaatagttattctctaagaggaaactagtgtttgttaaaaacaaaaataaaaacaaaaccacacaaggagaacccaattttgtttcaacaatttttgatcaatgtatatgaagctcttgataggacttccttaagcatgacgggaaaaccaaacacgttccctaatcaggaaaaaaaaaaaaaaaaaaaagtaagacacaaacaaaccatttttttctctttttttggagttgggggcccagggagaagggacaaggcttttaaaagacttgttagccaacttcaagaattaatatttatgtctctgttattgttagttttaagccttaaggtagaaggcacatagaaataacatc

SEQ ID NO: 4 - protein, 894 aa, Homo sapiens
MILLAVLFLCFISSYSASVKGHTTGLSLNNDRLYKLTYSTEVLLDRGKGKLQDSVGYRISSNVDVALLWRNPDGDDDQLIQITMKDVNVENVNQQRGEKSIFKGKSPSKIMGKENLEALQRPTLLHLIHGKVKEFYSYQNEAVAIENIKRGLASLFQTQLSSGTTNEVDISGNCKVTYQAHQDKVIKIKALDSCKIARSGFTTPNQVLGVSSKATSVTTYKIEDSFVIAVLAEETHNFGLNFLQTIKGKIVSKQKLELKTTEAGPRLMSGKQAAAIIKAVDSKYTAIPIVGQVFQSHCKGCPSLSELWRSTRKYLQPDNLSKAEAVRNFLAFIQHLRTAKKEEILQILKMENKEVLPQLVDAVTSAQTSDSLEAILDFLDFKSDSSIILQERFLYACGFASHPNEELLRALISKFKGSIGSSDIRETVMIITGTLVRKLCQNEGCKLKAVVEAKKLILGGLEKAEKKEDTRMYLLALKNALLPEGIPSLLKYAEAGEGPISHLATTALQRYDLPFITDEVKKTLNRIYHQNRKVHEKTVRTAAAAIILNNNPSYMDVKNILLSIGELPQEMNKYMLAIVQDILRLEMPASKIVRRVLKEMVAHNYDRFSRSGSSSAYTGYIERSPRSASTYSLDILYSGSGILRRSNLNIFQYIGKAGLHGSQVVIEAQGLEALIAATPDEGEENLDSYAGMSAILFDVQLRPVTFFNGYSDLMSKMLSASGDPISVVKGLILLIDHSQELQLQSGLKANIEVQGGLAIDISGAMEFSLWYRESKTRVKNRVTVVITTDITVDSSFVKAGLETSTETEAGLEFISTVQFSQYPFLVCMQMDKDEAPFRQFEKKYERLSTGRGYVSQKRKESVLAGCEFPLHQENSEMCKVVFAPQPDSTSSGWF

SEQ ID NO: 5 - DNA, 2878 bp, Mus musculus
ctggatgtggcagagggagccagcatgatcctcttggcagtgctttttctctgcttcttctcctcctactctgcttccgttaaaggtcacacaactggcctctcattaaataatgagcggctatacaagctcacgtactccactgaagtgtttcttgatgggggcaaaggaaaaccgcaagacagcgtgggctacaaaatctcatctgatgtggacgttgtgttactgtggaggaatcctgatggtgatgatgatcaagtgatccaagtcacgataacagctgttaacgttgaaaatgcgggtcaacagagaggcgagaagagcatcttccagggcaaaagtacacctaagatcatagggaaggacaacctggaggctctgcagagacccatgcttcttcatctggtccgggggaaggtcaaggagttctactcctatgaaaacgagccagtgggcatagaaaatctcaagagaggcttggctagcttattccagatgcagctaagctctggaactaccaacgaggtagatatctctggggattgtaaagtgacctaccaggcccaacaagacaaagtggtcaaaattaaggctctggatacatgcaaaattgagcggtctggatttacaacggcaaaccaggtgctgggcgtcagttcaaaagccacatctgtcactacctacaagatagaggacagctttgtcaccgctgtgcttgcagaagagaccagggcttttgccttgaacttccaacaaaccatagcaggaaaaatagtgtcaaagcagaaattggagctgaagacaactgaagccggcccaaggatgatccccgggaagcaagtggcaggtgtaattaaagcagttgattccaaatacaaagccattcccattgtgggacaggtcctcgagcgtgtctgcaaaggatgcccttctctggcggagcactggaagtccatcagaaagaacctggagcctgaaaacctgtccaaggccgaggctgtccagagcttcctggccttcatccagcacctccggacttcgaggagagaagagatcctccagattctgaaggcagagaagaaagaagtgctccctcagctggtggatgccgtcacctctgctcagactccagactcgctagaagccatcctggactttttggatttcaaaagtgacagcagtatcatactccaggaaaggttcctctatgcctgtggctttgccacccaccctgatgaagaactcctacgagccctccttagtaagttcaaaggttcctttgcaagcaacgacatcagagagtcggttatgatcatcattggagccctagtcaggaagctgtgtcagaatgaaggctgcaagctcaaggcagtggtggaagctaagaagctgatcctgggaggacttgaaaaaccagagaagaaagaagacaccacaatgtacctgctggccctgaagaatgccttgcttcccgaaggcatcccgctccttctgaagtatgctgaggctggagaagggcccgtcagccacctggccaccactgttctccagagatacgatgtctccttcatcacagatgaggtgaagaagaccttgaacaggatataccaccagaatcgtaaggttcatgagaagacggtgcgcacaactgccgctgctgtcatcttaaagaacccatcctacatggatgtgaagaacatcctgctgtccattggggaactcccgaaagagatgaacaaatacatgctcaccgttgtgcaagacatcctgcattttgaaatgcctgcaagcaaaatgatccgtcgagttctcaaggagatggctgttcacaattatgaccgtttctccaagagtggatcctcttctgcctatactggctacgtagaacgtagcccccgtgcagcgtccacatacagccttgacatcctttactctggctctggcattctgaggagaagtaacctgaacatcttccagtacatcaaaggaacagagcttcatggtagtcaggtggtgattgaagcccaagggctggaaggcttaattgcagccactcctgatgaaggagaggagaaccttgactcttatgctggcatgtcagccatcctgtttgatgttcagcttaggcctgtcacattttttaatggatacagtgatttgatgtccaaaatgctgtcggcatccggcgaccctgtcagcgtggtgaaagggcttattctgttaatagaccattctcaggatattcagctgcaatctggactaaaggccaatatggagatccagggtggtctagctattgatatttctggttcaatggaattcagtctgtggtatcgcgagtctaaaacccgagtgaaaaatcgggtggctgtggtgataaccagcgacgtcacagtggatgcctcttttgtgaaagctggtctggaaagcagagcggagacagaggctgggctggagttcatctccacagtgcagttctcacagtacccgttcttggtctgcatgcagatggacaaggctgaagccccactcaggcaattcgagacaaagtatgaaaggctatctacaggcaggggatatgtctctcggagaagaaaagagagcctagtggccggatgtgaactccccctccatcaacagaactctgagatgtgcaacgtggtattcccacctcagccagaaagcgataactccggtggatggttttgattcccgtgggttcccttccaccagaacgatatgctatgacgtgcctgacccttgctctctgagagcacagtgtttacatatttacctgtatttaagatgtttgtaaagagcagtggagaacttcagttgattaaagttgaacctattcaggagaagacccacagtgtcc

SEQ ID NO: 6 - protein, 894 aa, Mus musculus
MILLAVLFLCFFSSYSASVKGHTTGLSLNNERLYKLTYSTEVFLDGGKGKPQDSVGYKISSDVDVVLLWRNPDGDDDQVIQVTITAVNVENAGQQRGEKSIFQGKSTPKIIGKDNLEALQRPMLLHLVRGKVKEFYSYENEPVGIENLKRGLASLFQMQLSSGTTNEVDISGDCKVTYQAQQDKVVKIKALDTCKIERSGFTTANQVLGVSSKATSVTTYKIEDSFVTAVLAEETRAFALNFQQTIAGKIVSKQKLELKTTEAGPRMIPGKQVAGVIKAVDSKYKAIPIVGQVLERVCKGCPSLAEHWKSIRKNLEPENLSKAEAVQSFLAFIQHLRTSRREEILQILKAEKKEVLPQLVDAVTSAQTPDSLEAILDFLDFKSDSSIILQERFLYACGFATHPDEELLRALLSKFKGSFASNDIRESVMIIIGALVRKLCQNEGCKLKAVVEAKKLILGGLEKPEKKEDTTMYLLALKNALLPEGIPLLLKYAEAGEGPVSHLATTVLQRYDVSFITDEVKKTLNRIYHQNRKVHEKTVRTTAAAVILKNPSYMDVKNILLSIGELPKEMNKYMLTVVQDILHFEMPASKMIRRVLKEMAVHNYDRFSKSGSSSAYTGYVERSPRAASTYSLDILYSGSGILRRSNLNIFQYIKGTELHGSQVVIEAQGLEGLIAATPDEGEENLDSYAGMSAILFDVQLRPVTFFNGYSDLMSKMLSASGDPVSVVKGLILLIDHSQDIQLQSGLKANMEIQGGLAIDISGSMEFSLWYRESKTRVKNRVAVVITSDVTVDASFVKAGLESRAETEAGLEFISTVQFSQYPFLVCMQMDKAEAPLRQFETKYERLSTGRGYVSRRRKESLVAGCELPLHQQNSEMCNVVFPPQPESDNSGGWF

SEQ ID NO: 7 - DNA, 25 bp, Artificial Sequence
  Description of Artificial Sequence oligonucleotide primer
ggactttttggatttcaaaagtgac

SEQ ID NO: 8 - DNA, 21 bp, Artificial Sequence
  Description of Artificial Sequence oligonucleotide primer
ggagaaacggtcataattgtg

SEQ ID NO: 9 - DNA, 21 bp, Artificial Sequence
  Description of Artificial Sequence oligonucleotide primer
gtgggccgctctaggcaccaa

SEQ ID NO: 10 - DNA, 24 bp, Artificial Sequence
  Description of Artificial Sequence oligonucleotide primer
ctctttgatgtcacgcacgatttc

SEQ ID NO: 11 - DNA, 25 bp, Artificial Sequence
  Description of Artificial Sequence oligonucleotide primer
ggactttttggatttcaaaagtgac

SEQ ID NO: 12 - DNA, 21 bp, Artificial Sequence
  Description of Artificial Sequence oligonucleotide primer
ggagaaacggtcataattgtg

SEQ ID NO: 13 - DNA, 32 bp, Artificial Sequence
  Description of Artificial Sequence oligonucleotide primer
atctggcaccacaccttctacattgagctgcg

SEQ ID NO: 14 - DNA, 32 bp, Artificial Sequence
  Description of Artificial Sequence oligonucleotide primer
cgtcatactcctgcttgctgatccacatctgc

SEQ ID NO: 15 - protein, 264 aa, Homo sapiens
  VARIANT (1)..(264): wherein Xaa is any amino acid
MDPPRPALLALLAXPXLLLLLLAGARXEEEXLENVXLVCPKDXTRFXHLXKXXYNYEAESSSGVPGTAXSRSATRXNCKXELEVPQLCSFILKXSQCTLKEVYGFNPEGKALLKKTKNSXEXAAAMSRXELKLAIPEGKQVFLYPEKDEPTYILNIKRGIISALLVPPEXEEAKQXLFXDTVYGNCSTHFTVKTRXGNXATXXSTERDLGQCDRFKPIRTGISPXALIKGMXRPLSTLIXSXQSCQXTLDAKRKHVAEXXCKEQ

SEQ ID NO: 16 - protein, 335 aa, Homo sapiens
  VARIANT (1)..(335): wherein Xaa is any amino acid
MGCLLFLLLWALLQAWGSAEVPQRLFPLRCLQXSXFANSSWTRTDXLAWXGELQXHSWSNDXDTVXSLXPXXQGTFSDQQWETLQHIFRVYRSSFTRDVKXFAKMLRXSYPXELQVSAGCEVHPGNAXNNFFHVAFQGKDILSFQGTSWEPTQEAPLWVNLAIQVLNQXKXTRETVQXLLNGTCPQFVSGLLESGKSELKKQVKPKAWLSRGPXPGPGRXLLXCHVSGFYPKPVWVKWMXGEQEQQGTQPGDXLPNADETWYLRATLXXVAGEAAGLSCRVKHSSLEGQDXVLYWGGXYTSMGLIXLXVLACLLFLLIVGXTSRFKRQTSYQXXL

SEQ ID NO: 17 - RNA, 21 nt, Artificial
  Description of Artificial Sequence chemically synthesized siRNA
nnuuaugaccguuucuccagg

SEQ ID NO: 18 - RNA, 21 nt, Artificial Sequence
  Description of Artificial Sequence chemically synthesized siRNA
aagcucacguacuccacugaa

SEQ ID NO: 19 - protein, 210 aa, Homo sapiens
  VARIANT (1)..(210): wherein Xaa is any amino acid
KCVQSXKPSLMIQKAXXQALRKMEPKDKDQEVLLQTFLDDASPGDXRXAAXLMXXRSPSQADXNKIVQXLPWEQNEQVKNXVAXHIANXLNSEEXDXQDLKKLVXEAXKESQLPTVMDFRKFSRNYQLYKSVXLPSLDPXSXKIEGNLXFDPNNXLPKESMXXTTLTAFGFASXDXXEIXLEGKGFEPTLXAXFGKQXFFPXSVNKALYW

SEQ ID NO: 20 - protein, 335 aa, Homo sapiens
  VARIANT (1)..(335): wherein Xaa is any amino acid
MGCLLFLLLWALLQAWGSAEVPQRLFPLRCLQISSFANSSWTRTDGLAWLGELQTHXWSNDSDTVRXXKPWSQGTFSDQQWETLQHIFRVYRSSFTXDXKEXAKXXRLSYPLELQXSAGCEXHPGNASNNFFHVAFQGKDILSFQGTSXEPXQEAPXWVNLAXQXLNQDKWTXETXQWLLNGTCPQFVSGLLESGKSELKKQVKPKXWLSRGPXPXPGRLLLXCHVSGXYPKPVWVKWXXGEQEQQGTQPXDXXPNXDETWYLRATLXVXAGEAXGLSCRVKHSSLXGQDIVLYWGGSYTSMGLIALAVLACLLFLLIVGFTSRFKRQTSYQGVL

SEQ ID NO: 21 - protein, 301 aa, Homo sapiens
  VARIANT (1)..(301): wherein Xaa is any amino acid
FSYNNKYGMVAQVTQTLKLEDTPKINSRFFGEGTXKMGLAXESTKSTSPPKXAEAVXXXLQELKKLTISXQXIQRAXLFNXXVTELRGLSDEAVTSXLPQLIEXSSPXXLQALVQCGXPQCSTHIXQXLKXVHANPLLIDVVTYLVALXPEPSAQQXREIFNMARXQRSRATLYALSHAVNNYHKXNPXGTQELXDIANXLMEQIQDDCXGDEDYTYLXLRXIGNMGQTMEQLTPELKSXILKCVQSTKPSXXIQKAAIQXLRKMEPKDKDQXXXLQTFLDDASPGDKRLAAYLMLXRSPS

SEQ ID NO: 22 - protein, 335 aa, Homo sapiens
  VARIANT (1)..(335): wherein Xaa is any amino acid
MGCLLFLLLWALLQAWGSAEVPQRLFPLRCLQISSFANSSWTXTDGLAXLGELQTHSWSXDSDTXXXLKPWSQGTFSXQXWETLXHIFXXYRSSFTRDVKEFAKXLRLSYPXELQXXAGCEVHPGXASNNFFHXAXQGXDILSFQGTSWEPTQEAPXWVNLAIQXLNQDKWTRXTVQWLLNGTCPQFVSGLLEXGKXELKKQXKPKAXLSRGPSPGPGRLLLVCHVXGFYPKPVWXKWXRGEQEQQGTQPGDILPNXDETWYLRATLDXXAGEAAGLXCRVKHSSLEGQXXXLYWGGSYTSMGLIALAVLACLXFLLIVGFTSRFKRQTSYQGVL